(19) United States
(12) Patent Application Publication
(10) Pub. No.: US 2025/0066407 A1
(43) Pub. Date: Feb. 27, 2025

(54) FREE-FLOWING CARBOHYDRATES AND PROCESS FOR THE PREPARATION THEREOF

(71) Applicant: Südzucker AG, Mannheim (DE)

(72) Inventors: Rainer Kliß, Obrigheim (DE); Jan Strack, Obrigheim (DE); Jörg Bernard, Obrigheim (DE)

(21) Appl. No.: 18/722,242

(22) PCT Filed: Dec. 20, 2022
(86) PCT No.: PCT/EP2022/087056

§ 371 Date: Jun. 20, 2024

(30) Foreign Application Priority Data
Dec. 21, 2021 (EP) 21216373.7

(51) Int. Cl. C07H 1/08; C07H 3/02; C07H 3/04

(52) U.S. Cl. CPC C07H 1/08; C07H 3/02; C07H 3/04

(57) ABSTRACT

The present invention relates to a process for preparing a dried carbohydrate preparation from a water-containing crystalline carbohydrate starting preparation and carbohydrate particles, and to the carbohydrate preparation prepared by the process.

The present invention relates to a process for preparing a dried carbohydrate preparation from a water-containing crystalline carbohydrate starting preparation and carbohydrate particles.

Crystalline carbohydrate preparations which are coated on the surface with a syrup film or partially amorphous solid carbohydrate preparations tend to absorb water during storage and subsequently stick together. Such a syrup film can originate from previous process steps of the carbohydrate processing. This leads to caked carbohydrate preparations that are not or badly free-flowing, transportable, meterable and storeable. For marketable carbohydrate preparations, however, it is necessary to provide carbohydrate preparations that are stable in storage and meterable. All process steps of conveying, weighing, Metering and similar mechanical treatments pose a problem when processing easily clumping, non-free-flowing solid carbohydrate preparations. However, solid crystalline carbohydrate preparations that are less purified or coated with a syrup film with desired flavour properties, for example for an improved aroma, are generally particularly appreciated, at least by certain consumer groups. However, so-called ‘soft-sugar carbohydrate preparations’ (such as ‘farin sugar’ or ‘basterd sugar’), i.e. sticky carbohydrate preparations, are only actually accepted by buyers as a speciality in specific individual cases and in local markets. As a rule, however, the buyer, both the end consumer and the processor, insists on carbohydrate preparations that are free-flowing and stable in storage. In particular, ‘soft-sugar carbohydrate preparations’ pose problems for processors in terms of handling and packaging, with economically unfavourable packaging times and frequent production disruptions. In addition, the carbohydrate preparations should contain as few additives or excipients as possible. This also includes the use of so-called free-flow agents.

Prior art carbohydrate preparations are often dried to such an extent that a syrup film adhering to the crystals no longer tends to stick directly. In such cases, hermetically sealed packaging is necessary to prevent subsequent water absorption through a sorption procedure. However, not all carbohydrate preparations can be treated in this way. The main problem with this method is the complex packaging and storage, as this must prevent subsequent water absorption. Badly dryable carbohydrate preparations can be converted into a dry form using a spray-drying process. However, this technique is costly and is not suitable for carbohydrate preparations in which crystals dominate and only a small amount of syrup is present. This method also requires elaborate packaging and storage to prevent subsequent water absorption and sticking. For crystalline carbohydrate preparations whose surfaces tend to stick together, so-called flow agents can be used. These typically do not consist of the same carbohydrates of which the desired end product consists of, but belong to the class of inorganic substances (such as SiO₂, blood lye salt) or starches. This has the disadvantage that additional excipients must be declared in the carbohydrate preparation, which can pose a regulatory hurdle or may not be accepted by consumers. A slight sensory influence cannot be ruled out either.

A disadvantage of certain carbohydrate preparations, in particular crystalline sucrose-containing carbohydrate preparations, is that an increased water content (>0.05 wt.-%, based on the total weight of the crystalline, in particular sucrose-containing, carbohydrate preparation), in particular if there is a syrup film on the crystalline particles, clumping and a loss of free-flowability appears. In addition, substances remaining on the crystal surface can increase hygroscopicity.

The technical problem underlying the invention therefore lies in overcoming the aforementioned disadvantages. In particular, the technical problem underlying the invention is to provide inexpensive and easy to carry out processes which make it possible to provide a carbohydrate preparation which is free-flowing, transportable, meterable and storable, in particular from a carbohydrate starting preparation which has syrup film-coated crystalline particles, wherein the carbohydrate preparation obtained is provided in particular with as few as possible to no additives or excipients, in particular flow agents, and in particular is easy to pack.

In particular, the technical problem underlying the present invention is to provide a process for drying a crystalline carbohydrate starting preparation dissolved in water, which has at least two carbohydrates with different water solubility, in particular at least one poorly-soluble carbohydrate and at least one well-soluble carbohydrate, which leads the provision of dried carbohydrate preparations efficiently and with as little disturbance as possible and in particular enables the drying of carbohydrate preparations which are difficult or impossible to dry using conventional processes.

The technical problem underlying the invention is solved by the teachings of the present invention, in particular by a process for preparing a dried carbohydrate preparation from a water-containing crystalline carbohydrate starting preparation, comprising the process steps:
a) providing a water-containing crystalline carbohydrate starting preparation having 0.4 to 22.5 wt.-%, in particular 2.5 to 12 wt.-% water (each based on the total weight of the carbohydrate starting preparation),), wherein the carbohydrate starting preparation is selected from the group consisting of a sucrose-containing composition an isomaltulose- and trehalulose-containing composition and an isomalt-containing composition in a drying reactor, and carbohydrate particles, wherein the carbohydrate particles have a proportion of carbohydrate crystals with a diameter of less than or equal to 100 μm of at least 80 wt.-%, in particular wherein the carbohydrate starting preparation has a content of water in excess of any possible water of crystallisation present, in particular in the form of a liquid-film on crystalline particles of the carbohydrate starting preparation,
b) metering-in the carbohydrate particles via a metering device assigned to the drying reactor into the water-containing crystalline carbohydrate starting preparation presented in the drying reactor in an amount of 2 to 30 wt.-% (based on the total weight of the carbohydrate starting preparation) at a pressure of 10 to 1100 mbar, wherein the water-containing carbohydrate starting preparation presented in the drying reactor has a temperature of 20 to 80° C., in particular 40 to 60° C., and
c) homogenising the obtained mixture by means of a mixing device under the conditions mentioned in process step b) to obtain a dried carbohydrate preparation.

According to the invention, preferably the water-containing crystalline carbohydrate starting preparation having 0.4 to 22.5 wt.-%, in particular 2.5 to 12 wt.-% water (each based on the total weight of the carbohydrate starting preparation) provided in process step a) has liquid-film-coated crystalline carbohydrate particles, in particular it consists of such liquid-film-coated crystalline particles. Preferably, in process step c), a dried carbohydrate preparation comprising liquid-film-freed crystalline carbohydrate particles, in particular consisting of such, is obtained.

The water-containing crystalline carbohydrate starting preparation used according to the invention is in particular a preparation in which the crystalline mass of the carbohydrates, in particular the crystalline particles, is coated with a liquid film, also referred to as liquid-film-coated in the present case. The liquid-film, also referred to as syrup film in the present case, represents an aqueous solution or suspension of the carbohydrate or carbohydrates of the starting preparation which is present on the surface of the crystalline particles, and may originate from previous process steps of the carbohydrate processing. The syrup film thus represents a water proportion that is not integrally present in the crystalline particle, but is present on the surface of the crystalline particles; in particular, the water proportion of the syrup film is not water of crystallisation. If water of crystallisation is present in a carbohydrate, the syrup film represents a further, i.e. additional, water proportion in the product. Preferably according to the invention, the water-containing crystalline carbohydrate starting preparation therefore has crystalline particles which have a water content in excess of any possible water of crystallisation present. The water-containing crystalline carbohydrate starting preparation used according to the invention is therefore not a dried carbohydrate preparation containing water of crystallisation or a dried carbohydrate preparation free of water of crystallisation.

Surprisingly, the metering-in of carbohydrate particles according to the invention, wherein the carbohydrate particles have a proportion of carbohydrate crystals with a diameter of less than or equal to 100 μm of at least 80 wt.-%, to the carbohydrate particles having a syrup film of the water-containing starting preparation does not lead to the expected clumping of crystalline particles, but rather exactly the opposite, to an advantageous increased free-flowability.

The technical problem underlying the invention is solved by the teachings of the present invention, in particular by a process for preparing a dried carbohydrate preparation from a water-containing crystalline carbohydrate starting preparation, comprising the process steps:
a) providing a water-containing crystalline carbohydrate starting preparation having 0.4 to 22.5 wt.-%, in particular 2.5 to 12 wt.-% water (each based on the total weight of the carbohydrate starting preparation), wherein the carbohydrate starting preparation is selected from the group consisting of a sucrose-containing composition an isomaltulose- and trehalulose-containing composition and an isomalt-containing composition in a drying reactor, and carbohydrate particles, wherein the carbohydrate particles have a proportion of carbohydrate crystals with a diameter of less than or equal to 100 μm of at least 80 wt.-%,
b) metering-in of the carbohydrate particles via a metering device assigned to the drying reactor into the water-containing crystalline carbohydrate starting preparation presented in the drying reactor in an amount of 2 to 30 wt.-% (based on the total weight of the carbohydrate starting preparation) at a pressure of 10 to 1100 mbar, wherein the water-containing carbohydrate starting preparation presented in the drying reactor has a temperature of 20 to 80° C., in particular 40 to 60° C., and
c) homogenising the obtained mixture by means of a mixing device under the conditions mentioned in process step b) to obtain a dried carbohydrate preparation, in particular with a water content of at most 6.0, in particular at most 5.0, in particular at most 1.9 wt.-%, in particular 0.01 to 0.70 wt.-% (each based on the total weight of the dried carbohydrate preparation).

The invention starts from a water-containing crystalline carbohydrate starting preparation having 0.4 to 22.5 wt.-% water (based on the total weight of the carbohydrate starting preparation), which is provided in process step a) in a drying reactor and is subjected to a process which, in particular using process steps b) and c), leads to obtaining a dried carbohydrate preparation, wherein the dried carbohydrate preparation has a lower water content than the water-containing crystalline carbohydrate starting preparation.

The water-containing crystalline carbohydrate starting preparation used according to the invention is selected from the group consisting of a sucrose-containing composition, an isomaltulose- and trehalulose-containing composition and an isomalt-containing composition. In process step a), carbohydrate particles, wherein the carbohydrate of the carbohydrate particles is preferably identical to the carbohydrate of the carbohydrate starting preparation, are also provided which have a proportion of carbohydrate crystals with a diameter of less than or equal to 100 μm of at least 80 wt.-%. In a process step b), the carbohydrate particles provided in process step a) are introduced via a metering device assigned to the drying reactor into the water-containing crystalline carbohydrate starting preparation provided in the drying reactor in an amount of 2 to 30 wt.-% (based on the total weight of the carbohydrate starting preparation) at a pressure of 10 to 1100 mbar, wherein the water-containing carbohydrate starting preparation presented in the drying reactor has a temperature of 20 to 80° C., and a mixture of carbohydrate particles and water-containing starting preparation is thus obtained. The mixture thus obtained is homogenised in a process step c) by means of a mixing device under the conditions mentioned in process step b) and a dried carbohydrate preparation, in particular with a water content of at most 6.0, in particular at most 5.0, in particular at most 1.9 wt.-% (each based on the total weight of the dried carbohydrate preparation), is obtained.

Carrying out process steps b) and c) according to the invention leads to a drying of the crystalline carbohydrate starting preparation used, which has a specific water content, that is to say to obtaining a dried carbohydrate preparation which has a lower water content than the water-containing crystalline carbohydrate starting preparation having 0.4 to 22.5 wt.-% water (based on the total weight of the carbohydrate starting preparation) used in process step a). Process steps b) and c) therefore always lead to a reduction of this water content and at the same time to a surprisingly significantly improved free-flowability, regardless of the specific water content of the crystalline carbohydrate starting preparation used. In particular, the dried carbohydrate preparation obtained according to process steps b) and c) stands out by the fact that it can be further treated in an optional subsequent conditioning step without further ado to form a dried and conditioned product which is particularly easy meterable and free-flowing, which would not be possible without the upstream drying according to steps b) and c) provided according to the invention.

In particular, the present invention makes it possible for a water-containing crystalline carbohydrate starting preparation, for example a sucrose-containing starting preparation coated with a syrup film, provided in process step a) in a drying reactor, not to be dried at all or only to be dried to an economically favourable degree of drying prior to provision according to process step a). According to the invention, preparations originating directly from a carbohydrate crystallisation process, for example crystal suspensions which still have residual water contents, in particular residual mother liquor contents, can thus be dried without costly drying in a process according to the invention by the addition of carbohydrate particles in an amount of 2 to 30 wt.-% (based on the total weight of the carbohydrate starting preparation) as provided in process step b).

Accordingly, the invention provides a process for preparing a dried carbohydrate preparation from a water-containing crystalline carbohydrate starting preparation and carbohydrate particles, wherein a water-containing crystalline carbohydrate starting preparation having 0.4 to 22.5 wt. % (based on the total weight of the carbohydrate starting preparation) provided in a drying reactor and carbohydrate particles are provided in a process step a) and the carbohydrate particles are metered into the crystalline water-containing carbohydrate starting preparation provided in the drying reactor in a process step b), wherein the mixture obtained is homogenised in a process step c) and a dried carbohydrate preparation is thus obtained with the aid of the carbohydrate particles metered-in into the water-containing crystalline carbohydrate starting preparation.

In an advantageous and particularly preferred manner, the process according to the invention results in a simple and efficient, in particular cost-efficient, process control. In particular, a dried carbohydrate preparation is obtained by the process according to the invention which is free-flowing, transportable, meterable and storable, in particular more free-flowing, transportable, meterable and storable than dried carbohydrate preparations known in the prior art. Preferably, the dried carbohydrate preparation obtained by the process according to the invention is easier to pack than dried carbohydrate preparations known in the prior art, which require, for example, hermetically sealed packaging. Preferably, the process according to the invention advantageously manages with as few as possible to no additives or excipients, in particular flow agents, to obtain a dried carbohydrate preparation.

Furthermore, the present invention is advantageous in that the carbohydrate preparation obtained does not stick together even if water is subsequently absorbed and the carbohydrate preparation obtained is thus stable in storage.

The present invention is also advantageous in that the use of additives or excipients can be avoided, wherein a stable in storage and meterable dried carbohydrate preparation is nevertheless obtained. Furthermore, the present invention is advantageous in that the storage stability of the carbohydrate preparation obtained from the process according to the invention is not bound to a complex packaging concept in order to avoid subsequent water absorption and thus clumping.

The present invention makes it possible to obtain a stable in storage dried carbohydrate preparation from a carbohydrate starting preparation that is difficult to dry by means of a low-energy process with comparatively simple technical effort compared to the prior art. Preferably, mixing and metering devices and a drying reactor, in particular a sealable drying reactor, are used. Preferably, a low negative pressure in the drying reactor is particularly suitable for drying according to the invention. Preferably, the present invention enables the carbohydrate starting preparation to be dried to comprise mixtures of well-soluble carbohydrates and poorly-soluble carbohydrates, which is advantageous in particular for sensory reasons. Preferably, the present invention also enables a better marketable dried carbohydrate preparation according to the invention to be obtained from a water-containing carbohydrate starting preparation which tends to crystallise, with few and simple process steps, compared to the prior art. Particularly preferably, a process according to the invention comprises only comparatively simple process steps, namely providing, metering, homogenising and optionally packaging. Compared to the prior art, the carbohydrate preparations according to the invention exhibit good storage stability and remain free-flowing even if water is subsequently absorbed by sorption. In particular, the process according to the invention makes it possible to prepare a dried carbohydrate preparation that is sensory comparable to a traditional soft-sugar carbohydrate preparation without the dried carbohydrate preparation having the same difficult-to-process properties.

In a preferred embodiment of the present invention, the process according to the invention is carried out continuously, semi-continuously or batch-wise.

In a preferred embodiment of the present invention, the drying reactor used in process steps a) and b) and preferably also process step c) is a container, in particular a container with at least one mixing device or a container which is configured to carry out, preferably regular, mixing movements, in particular is capable of carrying out rotating or swivelling movements, in particular wherein one or more installations are present in the container.

In a preferred embodiment of the present invention, the drying reactor used in process steps a) and b) is in particular
a) a vertically or substantially upright dryer with a cylindrical design, preferably with one or more vertically installed agitator shafts, or
b) a dryer with a cylindrical and conical proportion of the drying housing or a completely conical dryer, preferably with an agitator shaft arranged vertically, or
c) a horizontal or essentially horizontal or inclined dryer, preferably with one or more horizontally or horizontally arranged agitator shafts, or
d) a moving, i.e. rotating or asymmetrically moving dryer with or without installations.

In a preferred embodiment of the present invention, the useable drying reactors according to the invention can be provided with stationary or moving, for example rotating, installations, in particular choppers, for better mixing of the material to be dried.

In a preferred embodiment of the present invention, the dryers can be arranged vertically, horizontally or at any angle to the earth's surface.

In a preferred embodiment of the present invention, the dryers, in particular the dryer housings, may perform symmetrical or asymmetrical movements.

In a preferred embodiment of the present invention, the dryers may have a symmetrical, or asymmetrical design, or any combination thereof, in particular double or triple cylindrical design. Preferably, the dryers may in particular be configured as a round or ellipsoidal or conical cylinder.

In a preferred embodiment of the present invention, the dryers may have one or more agitator shafts.

In a preferred embodiment of the present invention, the agitator shafts may have a fixed axis of rotation or moving position.

In a preferred embodiment of the present invention, the dryers may be operated in a vacuum or at a slight positive pressure.

In a preferred embodiment of the present invention, the dryers are provided with heating and/or cooling systems to adjust or control a certain temperature.

In a preferred embodiment of the present invention, the water-containing crystalline carbohydrate starting preparation provided in process step a) has 0.4 to 22.5 wt.-%, in particular 0.5 to 22.5 wt.-%, in particular 0.7 to 20.0 wt.-%, in particular 1.0 to 18.0 wt.-%, in particular 1.5 to 15.0 wt.-%, in particular 2.0 to 13.0 wt.-%, in particular 2.5 to 12.0 wt.-%, in particular 4.0 to 10.0 wt.-%, in particular 6.0 to 8.0 wt.-%, in particular 0.4 to 4.0 wt.-%, in particular 0.4 to 3.5 wt.-%, in particular 0.4 to 3.0 wt.-%, in particular 0.4 to 2.5%, in particular 0.6 to 2.0 wt.-%, in particular 0.8 to 1.5 wt.-%, in particular 1.0 wt.-%, water (each based on the total weight of the carbohydrate starting preparation). This water content is present in the form of a liquid-film on the solid crystalline carbohydrate particles of the carbohydrate starting preparation. If the carbohydrate or carbohydrates of the carbohydrate starting preparation contain water of crystallisation, the water content is formed by the water content of the liquid-film and the water of crystallisation.

Particularly preferably, the water-containing crystalline carbohydrate starting preparation provided in process step a) is a crystalline mass of crystalline carbohydrate particles coated with a syrup film, in particular in solid or semi-solid form.

Particularly preferably, the water-containing crystalline carbohydrate starting preparation provided in process step a) is a suspension of crystalline carbohydrate particles in an aqueous medium, in particular a crystal magma.

In a preferred embodiment of the present invention, the water-containing carbohydrate starting preparation provided in process step a) comprises at least one poorly-soluble carbohydrate having a solubility at 20° C. in water of 5.0 to 53.0 g/100 g, in particular 14.5 to 53.0 g/100 g, in particular 15.2 to 53.0 g/100 g, in particular 29.0 to 53.0 g/100 g, in particular 47.1 to 53.0 g/100 g, in particular 5.0 g/100 g, in particular 14.5 g/100 g, in particular 15.2 g/100 g, in particular 29.0 g/100 g, in particular 47.1 g/100 g, and at least one well-soluble carbohydrate with a solubility at 20° C. in water of more than 53.0 g/100 g, in particular of at least 58.6 g/100 g, in particular of at least 66.7 g/100 g, in particular of at least 68.7 g/100 g, in particular of at least 70.0 g/100 g, in particular of at least 78.9 g/100 g.

In a preferred embodiment of the present invention, the water-containing carbohydrate starting preparation comprises at least one poorly-soluble carbohydrate selected from the group consisting of 1,1-GPM (1-O-α-D-glucopyranosyl-D-mannitol), isomaltulose, glucose and mannitol.

In a preferred embodiment of the present invention, the water-containing carbohydrate starting preparation comprises at least one well-soluble carbohydrate selected from the group consisting of sucrose, 1,6-GPS (6-O-α-D-glucopyranosyl-D-sorbitol), fructose, trehalulose and sorbitol.

In a preferred embodiment of the present invention, the water-containing carbohydrate starting preparation comprises at least one poorly-soluble carbohydrate selected from the group consisting of 1,1-GPM, isomaltulose, glucose and mannitol and one well-soluble carbohydrate selected from the group consisting of sucrose, 1,6-GPS, fructose, trehalulose and sorbitol.

In a preferred embodiment of the present invention, the poorly-soluble carbohydrate is glucose. In a preferred embodiment of the present invention, the glucose is present as a constituent of a carbohydrate-containing component selected from invert sugar, caramel sugar syrup and cane sugar syrup.

In a preferred embodiment of the present invention, the water-containing carbohydrate starting preparation comprises at least one poorly-soluble carbohydrate selected from the group consisting of 1,1-GPM, isomaltulose, glucose and mannitol with a solubility at 20° C. in water of 5.0 to 53.0 g/100 g, in particular 14.5 to 53.0 g/100 g, in particular 15.2 to 53.0 g/100 g, in particular 29.0 to 53.0 g/100 g, in particular 47.1 to 53.0 g/100 g, in particular 5.0 g/100 g, in particular 14.5 g/100 g, in particular 15.2 g/100 g, in particular 29.0 g/100 g, in particular 47.1 g/100 g, and at least one well-soluble carbohydrate selected from the group consisting of sucrose, 1,6-GPS, fructose, trehalulose and sorbitol with a solubility at 20° C. in water of more than 53.0 g/100 g, in particular of at least 58.6 g/100 g, in particular of at least 66.7 g/100 g, in particular of at least 68.7 g/100 g, in particular of at least 70.0 g/100 g, in particular of at least 78.9 g/100 g.

In a preferred embodiment of the present invention, the water-containing crystalline carbohydrate starting preparation provided in process step a) is a sucrose-containing starting preparation.

In a preferred embodiment of the present invention, the water-containing carbohydrate starting preparation provided in process step a) is a sucrose-containing starting preparation which has sucrose, preferably sucrose in crystalline form, in particular consists of it or contains at least one further substance.

In a preferred embodiment of the present invention, the water-containing carbohydrate starting preparation provided in process step a) is a sucrose-containing starting preparation which has sucrose, preferably sucrose in crystalline form, glucose and fructose, in particular consists of these.

In a preferred embodiment of the present invention, the water-containing crystalline carbohydrate preparation provided in process step a) comprises sucrose, in particular 91.0 to 99.5 wt.-%, in particular 92.0 to 98.5 wt.-%, in particular 92.5 to 98.2 wt.-%, in particular 92.7 to 98.1 wt.-%, in particular 93.0 to 98.1 wt.-% (each based on the total weight of the dry matter of the carbohydrate preparation provided in process step a)), glucose, in particular 0.30 to 1.00 wt.-%, 0.40 to 1.00 wt.-%, in particular 0.44 to 0.77 wt.-%, in particular 0.46 to 0.77 wt.-%, in particular 0.48 to 0.77 wt.-% (each based on the total weight of the dry matter of the carbohydrate preparation provided in process step a)), fructose, in particular 0.10 to 0.70 wt.-%, 0.30 to 0.60 wt.-%, in particular 0.41 to 0.55 wt.-%, in particular 0.43 to 0.54 wt.-%, in particular 0.44 to 0.50 wt.-% (each based on the total weight of the dry matter of the carbohydrate preparation provided in process step a)) and water, and optionally, minor components, in particular 0.40 to 4.50 wt.-%, in particular 0.40 to 4.36 wt.-%, in particular 0.42 to 4.24 wt.-%, in particular 0.40 to 4.04 wt.-% (each based on the total weight of the dry matter of the carbohydrate preparation provided in process step a)).

In a preferred embodiment of the present invention, the water-containing carbohydrate starting preparation provided in process step a) is a water-containing sucrose-containing composition comprising crystalline sucrose and at least one further carbohydrate-containing component selected from the group consisting of invert sugar, caramel sugar syrup and cane sugar syrup.

In a preferred embodiment of the present invention, the water-containing carbohydrate starting preparation provided in process step a) is a sucrose-containing starting preparation which has sucrose, preferably sucrose in crystalline form, glucose and fructose in the form of invert sugar syrup, in particular consists of these.

In a preferred embodiment of the present invention, the water-containing carbohydrate starting preparation provided in process step a) is a sucrose-containing starting preparation which has sucrose from sugar beet in crystalline form and sucrose in the form of caramel sugar syrup, in particular consists of these.

In a preferred embodiment of the present invention, the water-containing carbohydrate starting preparation provided in process step a) is a sucrose-containing starting preparation which has sucrose from sugar beet in crystalline form and sucrose from sugar cane in the form of cane sugar syrup, in particular consists of these.

In a preferred embodiment of the present invention, the water-containing sucrose-containing starting preparation provided in process step a) comprises sucrose, preferably sucrose in crystalline form, and invert sugar syrup or sucrose, preferably sucrose in crystalline form, and caramel sugar syrup or sucrose, preferably sucrose in crystalline form, and cane sugar syrup or sucrose, preferably sucrose in crystalline form, and cane sugar syrup and caramel sugar syrup.

In a preferred embodiment of the present invention, the water-containing sucrose-containing starting preparation provided in process step a) comprises sucrose from sugar beet, in particular consists of it.

In a preferred embodiment of the present invention, the water-containing sucrose-containing starting preparation provided in process step a) comprises sucrose from sugar cane, in particular consists of it.

In a preferred embodiment of the present invention, the water-containing sucrose-containing starting preparation provided in process step a) comprises sucrose, in particular sucrose from sugar beet and glucose or sucrose from sugar cane and glucose, in particular consists of these.

In a preferred embodiment of the present invention, the water-containing sucrose-containing starting preparation provided in process step a) comprises sucrose, in particular sucrose from sugar beet and fructose or sucrose from sugar cane and fructose, in particular consists of these.

In a preferred embodiment of the present invention, the water-containing sucrose-containing starting preparation provided in process step a) comprises sucrose, in particular sucrose from sugar beet and glucose and fructose or sucrose from sugar cane and glucose and fructose, in particular consists of these.

In a preferred embodiment of the present invention, the water-containing sucrose-containing starting preparation provided in process step a) comprises sucrose, in particular sucrose from sugar beet or sugar cane, glucose and/or fructose, in particular in the form of invert sugar syrup, sucrose in the form of caramel sugar syrup and sucrose from sugar cane in the form of cane sugar syrup, in particular consists of these.

In a particularly preferred embodiment of the present invention, the water-containing crystalline sucrose-containing starting preparation provided in process step a) comprises in particular 70.0 to 98.0 wt.-%, in particular 80 to 98.0 wt.-%, in particular 90.0 to 98.0 wt.-%, in particular 95.0 to 98.0 wt.-% of sucrose, in particular crystalline sucrose (each based on the total weight of the dry matter of the water-containing sucrose starting preparation).

In a particularly preferred embodiment of the present invention, the water-containing sucrose-containing starting preparation provided in process step a) comprises sucrose, in particular 80.0 to 98.0 wt.-%, in particular 90.0 to 98.0 wt.-%, in particular 95.0 to 98.0 wt.-% (each based on the total weight of the water-containing sucrose starting preparation), invert sugar syrup, in particular 1.40 to 12.0 wt.-%, in particular 1.40 to 6.0 wt.-%, in particular 1.40 to 3.0 wt.-% (each based on the total weight of the water-containing sucrose starting preparation) and caramel sugar syrup, in particular 0.60 to 8.0 wt.-%, in particular 0.6 to 4.0 wt.-%, in particular 0.6 to 2.0 wt.-% (each based on the total weight of the water-containing sucrose starting preparation), in particular consists of these.

In a particularly preferred embodiment of the present invention, the water-containing sucrose-containing starting preparation provided in process step a) comprises sucrose, in particular 80.0 to 92.2 wt.-%, in particular 85.0 to 92.2 wt.-%, in particular 90.0 to 92.2 wt.-% (each based on the total weight of the water-containing sucrose starting preparation), cane sugar syrup, in particular 1.8 to 4.6 wt.-%, in particular 1.80 to 3.4 wt.-%, in particular 1.8 to 2.40 wt.-% (each based on the total weight of the water-containing sucrose starting preparation) and caramel sugar syrup, in particular 6.00 to 15.4 wt.-%, in particular 6.0 to 11.6 wt.-%, in particular 6.0 to 7.6 wt.-% (each based on the total weight of the water-containing sucrose starting preparation), in particular consists of these.

In a particularly preferred embodiment, the present invention relates to a process as described above, wherein the water-containing crystalline carbohydrate starting preparation provided in process step a) is an isomalt starting preparation dissolved in water or an isomaltulose- and trehalulose-containing starting preparation dissolved in water.

In a particularly preferred embodiment, the present invention relates to a process as described above, wherein the water-containing crystalline carbohydrate starting preparation provided in process step a) is an isomalt starting preparation dissolved in water.

In a particularly preferred embodiment of the present invention, the isomalt is isomalt ST or isomalt GS.

In a particularly preferred embodiment of the present invention, the water-containing isomalt starting preparation provided in process step a) has a 1,1-GPM (1-O-alpha-D-glucopyranosyl-D-mannitol) and 1,6-GPS (6-O-alpha-D-glucopyranosyl-D-sorbitol) content of 90.0 to 100.0 wt.-%, in particular of 92.0 to 99.0 wt.-%, in particular of 93.0 to 98.0 wt.-%, in particular of 95.0 to 100.0 wt.-% (based on the total weight of the dry matter of the water-containing isomalt starting preparation).

In a particularly preferred embodiment of the present invention, the isomalt starting preparation provided in process step a) has a 1,1-GPM (1-O-alpha-D-glucopyranosyl-D-mannitol) content of 45.0 to 50.0 wt.-% and a 1,6-GPS (6-O-alpha-D-glucopyranosyl-D-sorbitol) content of 50 to 55 wt.-% (each based on the total weight of the dry matter of the water-containing isomalt starting preparation).

In a particularly preferred embodiment of the present invention, the isomalt starting preparation provided in process step a) has a 1,1-GPM (1-O-alpha-D-glucopyranosyl-D-mannitol) content of 20.0 to 30.0 wt.-% and a 1,6-GPS (6-O-alpha-D-glucopyranosyl-D-sorbitol) content of 70.0 to 80.0 wt.-% (each based on the total weight of the dry matter of the water-containing isomalt starting preparation).

In a particularly preferred embodiment of the present invention, the isomalt starting preparation provided in process step a) has 1,1-GPS (1-O-alpha-D-glucopyranosyl-D-sorbitol), sorbitol, mannitol, or GPI (glucopyranosyl-iditol) or a mixture of two or more thereof.

In a particularly preferred embodiment of the present invention, the isomalt starting preparation provided in process step a) has sorbitol, mannitol, or GPI each in an amount of 0.00 to 0.20 wt.-%, in particular of 0.04 to 0.17 wt.-% (each based on the total weight of the dry matter of the water-containing isomalt starting preparation, determined by GC).

In a particularly preferred embodiment of the present invention, the isomalt starting preparation provided in process step a) has 1,1-GPS in an amount of 0.20 to 0.70, in particular of 0.30 to 0.60 wt.-% (each based on the total weight of the dry matter of the water-containing isomalt starting preparation, determined by GC).

In a particularly preferred embodiment of the present invention, the isomalt starting preparation provided in process step a) has a pH of 4.0 to 4.7, in particular 4.1 to 4.5.

In a particularly preferred embodiment, the present invention relates to a process as described above, wherein the carbohydrate starting preparation provided in process step a) is an isomaltulose- and trehalulose-containing starting preparation dissolved in water.

In a particularly preferred embodiment, the present invention relates to a process as described above, wherein the carbohydrate starting preparation provided in process step a) is an isomaltulose- and trehalulose-containing starting preparation dissolved in water, wherein its isomaltulose content is from 65.0 to 90.0 wt.-%, in particular from 70.0 to 90.0 wt.-%, in particular from 75.0 to 88.0 wt.-%, in particular from 75.0 to 85.0 wt.-%, and the trehalulose content is from 5.0 to 15.0 wt.-%, in particular from 6.5 to 13.0 wt.-%, in particular from 6.0 to 12.0 wt.-%, in particular from 7.0 to 10.0 wt.-% (each based on the total weight of the dry matter of the water-containing isomaltulose and trehalulose-containing starting preparation).

In a particularly preferred embodiment, the present invention relates to a process as described above, wherein the carbohydrate starting preparation provided in process step a) is an isomaltulose- and trehalulose-containing starting preparation dissolved in water, wherein its isomaltulose content is from 65.0 to 90.0 wt.-%, in particular from 70.0 to 90.0 wt.-%, in particular from 75.0 to 88.0 wt.-%, in particular from 75.0 to 85.0 wt.-% and the trehalulose content is from 5.0 to 15.0 wt.-%, in particular from 6.5 to 13.0 wt.-%, in particular from 6.0 to 12.0 wt.-%, in particular from 7.0 to 10.0 wt.-% (each based on the total weight of the dry matter of the water-containing isomaltulose- and trehalulose-containing starting preparation), and wherein it has fructose, glucose and sucrose, and optionally isomelecitose and isomaltose.

In a particularly preferred embodiment, the present invention relates to a process as described above, wherein the carbohydrate starting preparation provided in process step a) is an isomaltulose- and trehalulose-containing starting preparation dissolved in water, wherein its isomaltulose content is from 65.0 to 90.0 wt.-%, in particular from 70.0 to 90.0 wt.-%, in particular from 75.0 to 88.0 wt.-%, in particular from 75.0 to 85.0 wt.-% and the trehalulose content from 5.0 to 15.0 wt.-%, in particular from 6.5 to 13.0 wt.-%, in particular from 6.0 to 12.0 wt.-%, in particular from 7.0 to 10.0 wt.-%, and wherein it has a fructose content of 2.0 to 4.0 wt.-%, a glucose content of 1.0 to 3.0 wt.-% and a sucrose content of 0.1 to 12.0 wt.-%, in particular 0.2 to 3.0 wt.-% (each based on the total weight of the dry matter of the water-containing isomaltulose and trehalulose-containing starting preparation), and optionally isomelecitose and isomaltose.

In a preferred embodiment of the present invention, the water-containing crystalline carbohydrate starting preparation is partially crystalline or fully crystalline.

In a preferred embodiment of the present invention, in a process step a1) prior to process step a), the water-containing carbohydrate starting preparation is prepared from at least two different carbohydrates.

In a preferred embodiment of the present invention, the water-containing crystalline carbohydrate starting preparation is prepared in a process step a1) by mixing at least two carbohydrates, in particular two carbohydrates with different solubility, in particular at least one carbohydrate with good solubility and at least one carbohydrate with poor solubility.

In a preferred embodiment of the present invention, the water-containing crystalline carbohydrate starting preparation is prepared in a process step a1) at a temperature of 50 to 70° C., in particular 55 to 65, in particular 60° C.

In a preferred embodiment of the present invention, the at least two carbohydrates used in process step a1) are selected from the group consisting of sucrose, in particular sucrose from sugar beet or sugar cane, glucose and/or fructose, in particular in the form of invert sugar syrup, sucrose in the form of caramel sugar syrup and sucrose from sugar cane in the form of cane sugar syrup.

In a preferred embodiment of the present invention, the carbohydrates of the water-containing crystalline carbohydrate starting preparation and the carbohydrate particles provided in process step a) are identical carbohydrates.

In a preferred embodiment of the present invention, the carbohydrate particles provided in process step a) comprise a sucrose-containing composition, an isomaltulose- and trehalulose-containing composition and/or an isomalt-containing composition or consist of these.

In a preferred embodiment of the present invention, the carbohydrate particles consist of sucrose or isomaltulose and trehalulose or isomalt or a mixture thereof.

In a preferred embodiment of the present invention, the carbohydrate particles consist of sucrose, in particular in crystalline form, in particular in the form of a powder.

In a preferred embodiment of the present invention, the carbohydrate particles consist of isomaltulose and trehalulose, in particular in crystalline form, in particular in the form of a powder.

In a preferred embodiment of the present invention, the carbohydrate particles consist of isomalt, in particular in crystalline form, in particular in the form of a powder.

In a preferred embodiment of the present invention, the carbohydrate particles provided in process step a) have a proportion of at least 80 wt.-% of carbohydrate crystals with a diameter of less than or equal to 100 μm, in particular less than or equal to 80 μm, in particular less than or equal to 70 μm, in particular less than or equal to 60 μm, in particular less than or equal to 50 μm, in particular less than or equal to 40 μm, in particular less than or equal to 32 μm, in particular less than or equal to 20 μm, in particular less than or equal to 10 μm (each based on the total weight of the carbohydrate particles).

In a preferred embodiment of the present invention, the carbohydrate particles provided in process step a) have a proportion of at least 80 wt.-%, in particular at least 85 wt.-%, in particular at least 90 wt.-%, in particular at least 95 wt.-%, of carbohydrate crystals with a diameter of less than or equal to 100 μm.

In a preferred embodiment of the present invention, the carbohydrate particles provided in process step a) have a proportion of at least 80 wt.-%, in particular at least 85 wt.-%, in particular at least 90 wt.-%, in particular at least 95 wt.-%, of carbohydrate crystals with a diameter of less than or equal to 100 μm and a proportion of at least 70 wt.-% of carbohydrate particles with a diameter of at most 32 μm.

In a preferred embodiment of the present invention, the carbohydrate particles provided in process step a) have a proportion of at least 80 wt.-%, in particular at least 85 wt.-%, in particular at least 90 wt.-%, in particular at least 95 wt.-%, of carbohydrate crystals with a diameter of less than or equal to 50 μm.

In a preferred embodiment of the present invention, the carbohydrate particles provided in process step a) have a proportion of at least 80 wt.-%, in particular at least 85 wt.-%, in particular at least 90 wt.-%, in particular at least 95 wt.-%, of carbohydrate crystals with a diameter of less than or equal to 100 μm, in particular less than or equal to 80 μm, in particular less than or equal to 70 μm, in particular less than or equal to 60 μm, in particular less than or equal to 50 μm, in particular less than or equal to 40 μm, in particular less than or equal to 32 μm, in particular less than or equal to 20 μm, in particular less than or equal to 10 μm (each based on the total weight of the carbohydrate particles).

In a preferred embodiment of the present invention, the carbohydrate particles provided in process step a) are crystalline, preferably completely crystalline.

In a preferred embodiment of the present invention, the carbohydrate particles provided in process step a) have no or only minor amorphous structures.

In a preferred embodiment of the present invention, the carbohydrate particles provided in process step a) are metered in in process step b) in an amount of 2 to 30 wt.-%, in particular of 7 to 30 wt.-%, in particular of 10 to 30 wt.-%, in particular of 7 to 25 wt.-%, in particular of 10 to 25 wt.-%, in particular 7 to 20 wt.-%, in particular 10 to 25 wt.-%, in particular 7 to 15 wt.-%, in particular 10 to 15 wt.-%, in particular 7 to 10 wt.-% (each based on the total weight of the carbohydrate starting preparation).

In a preferred embodiment of the present invention, the carbohydrate particles provided in process step a) are metered in in process step b) in an amount of 2 to 25 wt.-%, in particular 5 to 20 wt.-%, in particular 7 to 15 wt.-% and in particular 8 to 10 wt.-% (each based on the total weight of the carbohydrate starting preparation).

In a preferred embodiment of the present invention, the carbohydrate particles provided in process step a) are metered in in process step b) in an amount of 4 to 25 wt.-%, in particular 4 to 20 wt.-%, in particular 4 to 15 wt.-% and in particular 4 to 10 wt.-% (each based on the total weight of the carbohydrate starting preparation).

In a preferred embodiment of the present invention, process step b) is carried out at a temperature of 20 to 35° C., in particular 20 to 30° C.

In a preferred embodiment of the present invention, the metering-in of the carbohydrate particles in process step b) is carried out in a drying reactor free from excess pressure and negative pressure under atmospheric pressure, in a vacuum drying reactor under reduced pressure, in particular 10 to 900 mbar, in particular 20 to 600 mbar, in particular 30 to 400 mbar, in particular 40 to 200 mbar, in particular 50 to 100 mbar or in particular 600 to 800 mbar, in particular 650 to 750 mbar, in particular 700 mbar, or is carried out in a pressure drying reactor under a pressure of atmospheric pressure up to 1100 mbar, in particular above atmospheric pressure up to 1100 mbar.

In a preferred embodiment of the present invention, the metering-in, in particular the metering-in via a metering device, in particular via rotating metering systems, vibrating or oscillating metering systems or via pneumatic metering systems, in particular at excess pressure or at negative pressure.

In a preferred embodiment of the present invention, the metering-in in process step b) is a mechanical metering-in via rotating metering systems, wherein the rotating metering system is a rotary valve, a rotary feeder or a screw conveyor.

In a preferred embodiment of the present invention, the metering-in in process step b) is a vibrating or oscillating metering-in via vibrating or oscillating metering systems, wherein the vibrating or oscillating metering system is a vibratory feeder, in particular a linear vibratory feeder, or a helical conveyor.

In a preferred embodiment of the present invention, the metering-in in process step b) is a pneumatic metering-in via pneumatic metering systems, wherein the pneumatic metering system is a flight conveying system, a dense phase conveying system, a push conveying system or a plug conveying system.

In a preferred embodiment of the present invention, at least one, in particular at least two, in particular at least three, in particular at least four, in particular at least 5 nozzles are attached to the pneumatic metering system. Preferably, the nozzles advantageously serve to improve the distribution of the fine crystalline carbohydrates.

If necessary, the metering-in can also be carried out manually controlled by means of a metering scoop or a metering spoon.

In a preferred embodiment of the present invention, the mixture obtained in process step b) comprises the carbohydrates sucrose, glucose, fructose and water.

In a preferred embodiment of the present invention, the mixture used in process step c) and obtained in process step b) has a temperature of 20 to 80° C., in particular 25 to 60° C., in particular 30° C. to 50° C., in particular 40° C. to 50° C., in particular 25 to 40° C.

In a preferred embodiment of the present invention, the dried carbohydrate preparation obtained in process step c) has a water content of at most 6.0 wt.-%, in particular at most 5.0 wt.-%, in particular at most 1.9 wt.-%, in particular at most 1.5 wt. %, in particular at most 1.0 wt.-%, in particular at most 0.8 wt.-%, in particular at most 0.7 wt.-%, in particular at most 0.6 wt.-%, in particular at most 0.5 wt.-%, in particular at most 0.4 wt.-%, in particular at most 0.3 wt.-%, in particular at most 0.2 wt.-%, in particular at most 0.1 wt.-%, in particular 0.01 to 6.00 wt.-%, in particular 0.01 to 5.00 wt.-%, in particular 0.01 to 0.70 wt.-%, in particular 0.05 to 6.00 wt.-%, in particular 0.05 to 5.00 wt.-%, in particular 0.05 to 0.60 wt.-%, in particular 0.10 to 0.50 wt.-%, in particular 0.20 to 0.40 wt.-%, in particular 0.20 to 0.30 wt.-%, in particular 0.12 wt.-%, in particular 0.13 wt.-%, in particular 0.27 wt.-%, in particular 0.29 wt.-%, in particular 0.31 wt.-%, in particular 0.34 wt.-%, in particular 0.42 wt.-%, in particular 0.43 wt.-%, in particular 0.47 wt.-% (each based on the total weight of the dried carbohydrate preparation). Preferably, the water content of the dried carbohydrate preparation obtained in process step c) is solely the water of crystallisation present in the carbohydrate preparation.

According to the present invention, the dried carbohydrate preparation obtained in process step c) has a lower water content than the crystalline water-containing carbohydrate starting preparation provided in process step a).

In a preferred embodiment of the present invention, the crystalline water-containing carbohydrate starting preparation provided in process step a) has 0.4 to 22.5 wt.-%, in particular 0.5 to 22.5 wt.-%, in particular 0.7 to 20 wt.-%, in particular 1.0 to 18 wt.-%, in particular 1.5 to 15 wt.-%, in particular 1.5 to 15 wt.-%, in particular 1.5 to 15 wt.-%. %, in particular 1.5 to 15 wt.-%, in particular 2.0 to 13 wt.-%, in particular 2.5 to 12 wt.-%, in particular 4.0 to 10 wt.-%, in particular 6.0 to 8.0 wt.-%, in particular 0.4 to 4.0 wt.-%, in particular 0.4 to 3.5 wt.-%, in particular 0.4 to 3.0 wt.-%, in particular 0.4 to 2.5 wt.-%, in particular 0.6 to 2.0 wt.-%, in particular 0.8 to 1.5 wt.-%, in particular 1.0 wt.-%, water (each based on the total weight of the carbohydrate starting preparation) and the dried carbohydrate preparation obtained in process step c) has a water content of at most 6.0 wt.-%, in particular at most 5.0 wt.-%, in particular at most 1.9 wt.-%, in particular at most 1.5 wt.-%, in particular at most 1.0 wt.-%, in particular at most 0.8 wt.-%, in particular at most 0.7 wt.-%, in particular at most 0.6 wt.-%, in particular at most 0.5 wt.-%, in particular at most 0.4 wt.-%, in particular at most 0.3 wt.-%, in particular at most 0.2 wt.-%, in particular at most 0.1 wt.-%, in particular 0.01 to 6.00 wt.-%, in particular 0.01 to 5.00 wt.-%, in particular 0.01 to 0.70 wt.-%, in particular 0.05 to 6.00 wt.-%, in particular 0.05 to 5.00 wt.-%, in particular 0.05 to 0.60 wt.-%, in particular 0.10 to 0.50 wt.-%, in particular 0.20 to 0.40 wt.-%, in particular 0.20 to 0.30 wt.-%, in particular 0.12 wt.-%, in particular 0.13 wt.-%, in particular 0.27 wt.-%, in particular 0.29 wt.-%, in particular 0.31 wt.-%, in particular 0.34 wt.-%, in particular 0.42 wt.-%, in particular 0.43 wt.-%, in particular 0.47 wt.-% (each based on the total weight of the dried carbohydrate preparation), wherein the dried carbohydrate preparation obtained in process step c) has a lower water content than the aqueous crystalline carbohydrate starting preparation prepared in process step a). Preferably, the water content of the dried carbohydrate preparation obtained in process step c) is solely the water of crystallisation present in the carbohydrate preparation.

In a preferred embodiment of the present invention, the dried carbohydrate preparation obtained in process step c) comprises sucrose, glucose, fructose and water.

In a preferred embodiment of the present invention, the dried carbohydrate preparation obtained in process step c) comprises sucrose, in particular 92.0 to 98.5 wt.-%, in particular 92.5 to 98.2 wt.-%, in particular 92.7 to 98.1 wt.-%, in particular 93.0 to 98.1 wt.-% (each based on the total weight of the dried carbohydrate preparation obtained in process step c)), glucose, in particular 0.40 to 1.00 wt.-%, in particular 0.44 to 0.74 wt.-%, in particular 0.46 to 0.72 wt.-%, in particular 0.48 to 0.69 wt.-% (each based on the total weight of the dried carbohydrate preparation obtained in process step c)) fructose, in particular 0.30 to 0.50 wt.-%, in particular 0.41 to 0.48 wt.-%, in particular 0.43 to 0.47 wt.-%, in particular 0.44 to 0.45 wt.-% (each based on the total weight of the dried carbohydrate preparation obtained in process step c)) and water, in particular 0.40 to 6.00 wt.-%, in particular 0.40 to 5.00 wt.-%, in particular 0.40 to 2.0 wt.-%, in particular 0.58 to 1.87 wt.-%, in particular 0.61 to 1.82 wt.-%, in particular 0.63 to 1.74 wt.-%, in particular 0.33 to 0.49 wt.-%, in particular 0.37 to 0.46 wt.-%, in particular 0.44 to 0.45 wt.-% (each based on the total weight of the dried carbohydrate preparation obtained in process step c)). Preferably, the water content of the dried carbohydrate preparation obtained in process step c) is solely the water of crystallisation present in the carbohydrate preparation.

In a preferred embodiment of the present invention, the dried carbohydrate preparation obtained in process step c) comprises sucrose, in particular 92.0 to 98.5 wt.-%, in particular 92.5 to 98.2 wt.-%, in particular 92.7 to 98.1 wt.-%, in particular 93.0 to 98.1 wt.-% (each based on the total weight of the dried carbohydrate preparation obtained in process step c)), glucose, in particular 0.40 to 1.00 wt. %, in particular 0.44 to 0.74 wt.-%, in particular 0.46 to 0.72 wt.-%, in particular 0.48 to 0.69 wt.-% (each based on the total weight of the dried carbohydrate preparation obtained in process step c)), fructose, in particular 0.30 to 0.50 wt.-%, in particular 0.41 to 0.48 wt.-%, in particular 0.43 to 0.47 wt.-%, in particular 0.44 to 0.45 wt.-% (each based on the total weight of the dried carbohydrate preparation obtained in process step c)), minor components, in particular 0.40 to 4.50 wt.-%, in particular 0.40 to 4.36 wt.-%, in particular 0.42 to 4.24 wt.-%, in particular 0.40 to 4.04 wt.-% (each based on the total weight of the dried carbohydrate preparation obtained in process step c)) and water, in particular 0.40 to 6.00 wt.-%, in particular 0.40 to 5.00 wt.-%, in particular 0.40 to 2.0 wt.-%, in particular 0.58 to 1.87 wt.-%, in particular 0.61 to 1.82 wt.-%, in particular 0.63 to 1.74 wt.-%, in particular 0.33 to 0.49 wt.-%, in particular 0.37 to 0.46 wt.-%, in particular 0.44 to 0.45 wt.-% (each based on the total weight of the dried carbohydrate preparation obtained in process step c)). Preferably, the water content of the dried carbohydrate preparation obtained in process step c) is solely the water of crystallisation present in the carbohydrate preparation.

In a preferred embodiment of the present invention, the dried carbohydrate preparation obtained in process step c) has an angle of repose of 30.0 to 45.0°, in particular 38.0 to 42.0°, in particular 39.0 to 41.0°, in particular 38.1°, in particular 40.7°, in particular 41.9°.

In a preferred embodiment of the present invention, the dried carbohydrate preparation obtained in process step c) has a free-flowability of 0.1 to 45.0 s/100 g, in particular 0.2 to 45.0 s/100 g, in particular 0.5 to 40.0 s/100 g, in particular 1.5 to 35.0 s/100 g, in particular 2.0 to 25.0 s/100 g.

In a preferred embodiment of the present invention, the dried carbohydrate preparation obtained in process step c) has a free-flowability of 20.0 to 45.0 s/100 g, in particular 30.0 to 40.0 s/100 g, in particular 32.0 to 40.0 s/100 g, in particular 34.0 to 40.0 s/100 g, in particular 34.2 s/100 g, in particular 39.1 s/100 g, at an outlet opening size of 6 mm of the funnel used for the test.

In a preferred embodiment of the present invention, the dried carbohydrate preparation obtained in process step c) has a free-flowability of 10.0 to 25.0 s/100 g, in particular 12.0 to 23.0 s/100 g, in particular 14.0 to 18 s/100 g, in particular 15.5 s/100 g, in particular 15.7 s/100 g, in particular 22.5 s/100 g, at an outlet opening size of 8 mm of the funnel used for the test.

In a preferred embodiment of the present invention, the dried carbohydrate preparation obtained in process step c) has a free-flowability of 6.0 to 20.0 s/100 g, in particular 7.0 to 19.0 s/100 g, in particular 8.0 to 15.0 s/100 g, in particular 8.2 s/100 g, in particular 8.3 s/100 g, in particular 13.2 s/100 g, in particular 18.1 s/100 g, at an outlet opening size of 10 mm of the funnel used for the test.

In a preferred embodiment of the present invention, the dried carbohydrate preparation obtained in process step c) has a free-flowability of 3.0 to 15.0 s/100 g, in particular 4.0 to 14.0 s/100 g, in particular 5.0 to 13.0 s/100 g, in particular 5.2 s/100 g, in particular 5.3 s/100 g, in particular 8.1 s/100 g, in particular 12.6 s/100 g, at an outlet opening size of 11.3 mm of the funnel used for the test.

In a preferred embodiment of the present invention, the dried carbohydrate preparation obtained in process step c) has a free-flowability of 1.0 to 8.0 s/100 g, in particular 1.5 to 7.0 s/100 g, in particular 2.0 to 6.5 s/100 g, in particular 2.2 s/100 g, in particular 2.3 s/100 g, in particular 3.9 s/100 g, in particular 6.5 s/100 g, at an outlet opening size of 15 mm of the funnel used for the test.

In a preferred embodiment of the present invention, the dried carbohydrate preparation obtained in process step c) has a free-flowability of 0.1 to 4.0 s/100 g, in particular 0.2 to 3.0 s/100 g, in particular 0.2 to 2.0 s/100 g, in particular 0.2 s/100 g, in particular 0.6 s/100 g, in particular 1.9 s/100 g, at an outlet opening size of 25 mm of the funnel used for the test.

In a preferred embodiment of the present invention, the dried carbohydrate preparation obtained in process step c) has a total water content of 0.1 to 0.5 g/100 g, in particular 0.2 to 0.5 g/100 g, in particular 0.3 to 0.5 g/100 g, in particular 0.33 to 0.46 g/100 g, in particular 0.33 g/100 g, in particular 0.37 g/100 g, in particular 0.44 g/100 g, in particular 0.46 g/100 g.

In a preferred embodiment of the present invention, the dried carbohydrate preparation obtained in process step c) has a surface water content of 0.2 to 0.5 g/100 g, in particular 0.3 to 0.5 g/100 g, in particular 0.31 to 0.43 g/100 g, in particular 0.31 g/100 g, in particular 0.34 g/100 g, in particular 0.42 g/100 g, in particular 0.43 g/100 g.

In a particularly preferred embodiment of the present invention, the mixing device used in process step c) serves to mix the mixture obtained in process step b) and thus to homogenise it, i.e. to distribute the mixed components as uniformly as possible. The mixing device therefore serves to generate a mechanical agitation of the obtained mixture.

In a preferred embodiment of the present invention, the mixing device in process step c) is a mixing device arranged in the drying reactor, in particular one or more agitator shafts.

In a preferred embodiment of the present invention, the mixing device in process step c) is integrally connected to the drying reactor or is a constituent thereof and is configured as a drying reactor configured to carry out, preferably, regular mixing movements, which is in particular capable of carrying out rotating or swivelling movements, in particular wherein one or more installations are present in the drying reactor.

In a particularly preferred embodiment, process steps a), b) and c) are carried out in a drying reactor which is configured as a drying reactor configured to carry out, preferably regular, mixing movements, in particular is capable of carrying out rotating or swivelling movements, in particular wherein one or more installations are present in the drying reactor. In a preferred embodiment, the drying reactor and the mixing device constitute a single device.

In a preferred embodiment of the present invention, process step c) is carried out at a temperature of 20 to 35° C., in particular 20 to 30° C.

In a preferred embodiment of the present invention, following process step c) a conditioning is carried out in a process step d).

In a preferred embodiment of the present invention, the conditioning according to process step d) is carried out at a temperature of at least 30° C., in particular 30 to 180° C., in particular 35 to 160° C., in particular 45 to 100° C., in particular 35 to 60° C., in particular 40 to 60° C., in particular 50 to 60° C.

In a preferred embodiment of the present invention, the conditioning according to process step d) is carried out at a pressure of 10 to 1100 mbar, in particular 10 to 1000 mbar, in particular 10 to 900 mbar, in particular 20 to 600 mbar, in particular 30 to 400 mbar, in particular 40 to 200 mbar, in particular 50 to 100 mbar.

In a preferred embodiment of the present invention, the conditioning carried out in process step d) is a drying, in particular an air drying, in particular at an air temperature of 85 to 95° C., in particular 90° C.

In a preferred embodiment of the present invention, the conditioned carbohydrate preparation obtained in process step d) has a water content of at most 6.0 wt.-%, in particular at most 5.0 wt.-%, in particular at most 1.9 wt.-%, in particular at most 1.5 wt.-%, in particular at most 1.0 wt.-%, in particular at most 0.8 wt.-%, in particular at most 0.7 wt.-%, in particular at most 0.6 wt.-%, in particular at most 0.5 wt.-%, in particular at most 0.4 wt.-%, in particular at most 0.3 wt.-%, in particular at most 0.2 wt.-%, in particular at most 0.1 wt.-%, in particular 0.01 to 6.00 wt.-%, in particular 0.01 to 5.00 wt.-%, in particular 0.01 to 0.70 wt.-%, in particular 0.05 to 6.00 wt.-%, in particular 0.05 to 5.00 wt.-%, in particular 0.05 to 0.60 wt.-%, in particular 0.10 to 0.50 wt.-%, in particular 0.20 to 0.40 wt.-%, in particular 0.20 to 0.30 wt.-%, in particular 0.12 wt.-%, in particular 0.13 wt.-%, in particular 0.27 wt.-%, in particular 0.29 wt.-%, in particular 0.31 wt.-%, in particular 0.34 wt.-%, in particular 0.42 wt.-%, in particular 0.43 wt.-%, in particular 0.47 wt.-% (each based on the total weight of the dried carbohydrate preparation). Preferably, the water content of the conditioned carbohydrate preparation obtained in process step d) is solely the water of crystallisation present in the carbohydrate preparation.

In a preferred embodiment of the present invention, the conditioned carbohydrate preparation obtained in process step d) comprises sucrose, in particular 94.0 to 99.0 wt.-%, in particular 94.0 to 98.7 wt.-%, in particular 94.1 to 98.7 wt.-%, in particular 94.2 to 98.6 wt.-%, in particular 94.3 to 98.5 wt.-% (each based on the total weight of the conditioned carbohydrate preparation obtained in process step d)), glucose, in particular 0.40 to 1.00 wt.-%, in particular 0.45 to 0.76 wt.-%, in particular 0.47 to 0.73 wt.-%, in particular 0.48 to 0.70 wt.-% (each based on the total weight of the conditioned carbohydrate preparation obtained in process step d)) fructose, in particular 0.30 to 0.50 wt.-%, in particular 0.41 to 0.49 wt.-%, in particular 0.43 to 0.47 wt.-%, in particular 0.44 to 0.45 wt.-% (each based on the total weight of the conditioned carbohydrate preparation obtained in process step d)) and water, in particular 0.10 to 6.00 wt.-%, in particular 0.10 to 5.00%, in particular 0.40 to 6.00 wt.-%, in particular 0.10 to 0.50 wt.-%, in particular 0.12 to 0.42 wt.-%, in particular 0.13 to 0.29 wt.-%, in particular 0.27 to 0.28 wt.-% (each based on the total weight of the conditioned carbohydrate preparation obtained in process step d)). Preferably, the water content of the dried carbohydrate preparation obtained in process step c) is solely the water of crystallisation present in the carbohydrate preparation.

In a preferred embodiment of the present invention, the conditioned carbohydrate preparation obtained in process step d) comprises sucrose, in particular 94.0 to 99.0 wt.-%, in particular 94.0 to 98.7 wt.-%, in particular 94.1 to 98.7 wt.-%, in particular 94.2 to 98.6 wt.-%, in particular 94.3 to 98.5 wt.-% (each based on the total weight of the conditioned carbohydrate preparation obtained in process step d)), glucose, in particular 0.40 to 1.0 wt.-%, in particular 0.45 to 0.76 wt.-%, in particular 0.47 to 0.73 wt.-%, in particular 0.48 to 0.70 wt.-% (each based on the total weight of the conditioned carbohydrate preparation obtained in process step d)) fructose, in particular 0.30 to 0.50 wt.-%, in particular 0.41 to 0.49 wt.-%, in particular 0.43 to 0.47 wt.-%, in particular 0.44 to 0.45 wt.-% (each based on the total weight of the conditioned carbohydrate preparation obtained in process step d)), minor components, in particular 0.20 to 4.50 wt.-%, in particular 0.40 to 4.43 wt.-%, in particular 0.42 to 4.30 wt.-%, in particular 0.40 to 4.1 wt.-% (each based on the total weight of the conditioned carbohydrate preparation obtained in process step d)) and water, in particular 0.10 to 0.50 wt.-%, in particular 0.12 to 0.42 wt.-%, in particular 0.13 to 0.29 wt.-%, in particular 0.27 to 0.28 wt.-% (each based on the total weight of the conditioned carbohydrate preparation obtained in process step d)).

In a preferred embodiment of the present invention, the conditioned carbohydrate preparation obtained in process step d) has an angle of repose of 30.0 to 45.0°, in particular 33.0 to 41.0° in particular 35.0 to 38.5°, in particular 36.0 to 37.0°, in particular 33.7°, in particular 35.1° in particular 36.4°, in particular 36.6°, in particular 38.5°, in particular 40.8°.

In a preferred embodiment of the present invention, the conditioned carbohydrate preparation obtained in process step d) has a free-flowability of 0 to 45.0 s/100 g, in particular 0.1 to 45.0 s/100 g, in particular 1.3 to 40.0 s/100 g, in particular 2.0 to 30.0 s/100 g, in particular 3.0 to 25.0 s/100 g.

In a preferred embodiment of the present invention, the conditioned carbohydrate preparation obtained in process step d) has a free-flowability of 20.0 to 45, 0 s/100 g, in particular 28.0 to 37.0 s/100 g, in particular 30.0 to 37.0 s/100 g, in particular 32.0 to 36.5 s/100 g, in particular 28.5 s/100 g, in particular 30.6 s/100 g, in particular 32.6 s/100 g, in particular 36.2 s/100 g, in particular 36.4 s/100 g, at an outlet opening size of 6 mm of the funnel used for the test.

In a preferred embodiment of the present invention, the conditioned carbohydrate preparation obtained in process step d) has a free-flowability of 10.0 to 20.0 s/100 g, in particular 12.0 to 18.0 s/100 g, in particular 14.0 to 16.0 s/100 g, in particular 12.6 s/100 g, in particular 14.7 s/100 g, in particular 14.8 s/100 g, in particular 15.0 s/100 g, in particular 15.1 s/100 g, in particular 17.4 s/100 g, at an outlet opening size of 8 mm of the funnel used for the test.

In a preferred embodiment of the present invention, the conditioned carbohydrate preparation obtained in process step d) has a free-flowability of 6.0 to 12.0 s/100 g, in particular 7.0 to 10.0 s/100 g, in particular 8.0 to 9.5 s/100 g, in particular 6.6 s/100 g, in particular 7.7 s/100 g, in particular 8.0 s/100 g, in particular 8.2 s/100 g, in particular 8.3 s/100 g, in particular 9.3 s/100 g, in particular 9.7 s/100 g, at an outlet opening size of 10 mm of the funnel used for the test.

In a preferred embodiment of the present invention, the conditioned carbohydrate preparation obtained in process step d) has a free-flowability of 3.5 to 7.0 s/100 g, in particular 5.0 to 6.0 s/100 g, in particular 4.1 s/100 g, in particular 5.0 s/100 g, in particular 5.3 s/100 g, in particular 5.4 s/100 g, in particular 6.1 s/100 g, at an outlet opening size of 11.3 mm of the funnel used for the test.

In a preferred embodiment of the present invention, the conditioned carbohydrate preparation obtained in process step d) has a free-flowability of 1.0 to 6.0 s/100 g, in particular 1.5 to 5.0 s/100 g, in particular 2.0 to 4.0 s/100 g, in particular 1.7 s/100 g, in particular 2.1 s/100 g, in particular 3.8 s/100 g, in particular 4.6 s/100 g, at an outlet opening size of 15 mm of the funnel used for the test.

In a preferred embodiment of the present invention, the conditioned carbohydrate preparation obtained in process step d) has a free-flowability of 0.0 to 4.0 s/100 g, in particular 0.0 to 3.0 s/100 g, in particular 0.1 s/100 g, in particular 1.3 s/100 g, in particular 2.5 s/100 g, at an outlet opening size of 25 mm of the funnel used for the test.

In a preferred embodiment of the present invention, the conditioned carbohydrate preparation obtained in process step d) has a total water content of 0.1 to 6.00 g/100 g, in particular of 0.1 to 5.00 g/100 g, in particular of 0.1 to 0.5 g/100 g, in particular 0.12 to 0.42 g/100 g, in particular 0.2 to 0.42 g/100 g, in particular 0.3 to 0.42 g/100 g, in particular 0.12 g/100 g, in particular 0.13 g/100 g, in particular 0.27 g/100 g, in particular 0.28 g/100 g, in particular 0.29 g/100 g, in particular 0.42 g/100 g. In particular, the total water content of the carbohydrate preparation obtained is formed solely by the water of crystallisation.

In a preferred embodiment of the present invention, the conditioned carbohydrate preparation obtained in process step d) has a surface water content of 0.050 to 0.100 g/100 g, in particular 0.050 to 0.090 g/100 g, in particular 0.060 to 0.080 g/100 g, in particular 0.070 to 0.080 g/100 g, in particular 0.054 g/100 g, in particular 0.067 g/100 g, in particular 0.076 g/100 g, in particular 0.084, in particular it has no surface water content.

In a preferred embodiment of the present invention, the water-containing sucrose-containing starting preparation, in particular the sucrose, was recovered from sugar cane.

In a preferred embodiment of the present invention, the water-containing sucrose-containing starting preparation, in particular the sucrose, was recovered from sugar beet.

In a preferred embodiment of the present invention, the water-containing isomalt-containing starting preparation has a 1,1-GPM (1-O-alpha-D-glucopyranosyl-D-mannitol) and 1,6-GPS (6-O-alpha-D-glucopyranosyl-D-sorbitol) content of 95.0 to 100.0 wt.-% (based on total dry mass of the water-containing isomalt-containing starting preparation).

In a preferred embodiment of the present invention, the water-containing isomalt-containing starting preparation has a 1,1-GPM (1-O-alpha-D-glucopyranosyl-D-mannitol) content of 45.0 to 50.0 wt.-% and a 1,6-GPS % and a 1,6-GPS (6-O-alpha-D-glucopyranosyl-D-sorbitol) content of 50 to 55 wt.-% (each based on the total dry mass of the water-containing isomalt-containing starting preparation).

In a preferred embodiment of the present invention, the water-containing isomalt-containing starting preparation has a 1,1-GPM (1-O-alpha-D-glucopyranosyl-D-mannitol) content of 20.0 to 30.0 wt.-% and a 1,6-GPS % and a 1,6-GPS (6-O-alpha-D-glucopyranosyl-D-sorbitol) content of 70.0 to 80.0 wt.-% (each based on the total dry mass of the water-containing isomalt-containing starting preparation).

In a preferred embodiment of the present invention, the water-containing isomalt-containing starting preparation has 1,1-GPS (1-O-alpha-D-glucopyranosyl-D-sorbitol), sorbitol, mannitol, or GPI or a mixture of two or more thereof.

In a preferred embodiment of the present invention, the water-containing isomaltulose and trehalulose-containing starting preparation has fructose, glucose, isomaltose, or isomelecitose, or a mixture of two or more thereof.

In a preferred embodiment of the present invention, after process step c) or process step d), the dried carbohydrate preparation is packaged in a process step x).

In the context of the present invention, a ‘water-containing crystalline carbohydrate starting preparation’ is understood to mean a carbohydrate preparation which has a water content of 0.4 to 22.5 wt.-%, in particular 2.5 to 12 wt.-% (based on the total weight of the carbohydrate starting preparation) and a carbohydrate content, wherein the carbohydrates are present at least partially, preferably completely, in crystalline form, in particular in a form suspended in the aqueous solution or coated by the aqueous solution. In a preferred embodiment, the water-containing carbohydrate starting preparation may be present in liquid form, in particular as a suspension, or in semi-liquid or solid form, in particular it is present in solid form. In a particularly preferred embodiment, the carbohydrates of the water-containing crystalline carbohydrate starting preparation are present as crystalline solid carbohydrate crystals covered by a liquid-film (also referred to as syrup film in the context of the present invention). The syrup film represents an aqueous solution or suspension of the carbohydrate or carbohydrates of the starting preparation which is present on the surface of the crystal particles of the carbohydrate starting preparation and may originate from previous process steps of the carbohydrate processing. The syrup film thus represents a water proportion that is not integrally present in the crystalline particle, but is present on the surface of the crystalline particles; in particular, the water proportion of the syrup film is not water of crystallisation. If water of crystallisation is present in a carbohydrate, the syrup film represents a further, i.e. additional, water proportion of the carbohydrate. A water-containing crystalline carbohydrate starting preparation of the present invention therefore has, in the case of water of crystallisation containing carbohydrates such as isomalt, in particular its 1,1-GPM component, or isomaltulose, a further, additional water content in the form of the liquid-film. A water-containing crystalline carbohydrate starting preparation of the present invention therefore has, in the case of water of crystallisation-free carbohydrates such as sucrose a water content in the form of the liquid-film.

The water content of the water-containing crystalline carbohydrate starting preparation is determined by subtracting the dry weight of the water-containing crystalline carbohydrate starting preparation, without taking into account any possible water of crystallisation present, from the total weight of the water-containing crystalline carbohydrate starting preparation.

In the case of carbohydrates which have no water of crystallisation, the water content of the water-containing crystalline carbohydrate starting preparation is from the water in which the crystalline carbohydrate particles are present, in particular by which they are coated, i.e. by the liquid-film.

In the case of carbohydrates which have water of crystallisation, the water content of the water-containing crystalline carbohydrate starting preparation is from the water in which the crystalline carbohydrate particles are present, in particular by which they are coated, i.e. the liquid-film, and additionally by the water of crystallisation.

Water of crystallisation is the term for the water that is incorporated in crystalline solids on fixed lattice sites in the crystal structure. The stoichiometric water of crystallisation content can be determined by X-ray structure analysis of the solid. The real water of crystallisation content of a carbohydrate can be determined from the difference between the total water content and the free water content. The total water content is measured by Karl Fischer titration and the proportion of free water by drying experiments.

The presence of a syrup film can also be detected, for example, by means of free-flowability, stickiness and/or caking tests, in particular according to the method section of the present examples, in which crystalline particles without a syrup film coating, in particular completely dried or overdried crystalline particles, are compared with the crystalline particles to be tested, which possibly have a syrup film, and a syrup film coating is detected by a significant deterioration in free-flowability, an increase in stickiness and/or an increased tendency to cake, in particular all three.

Overdried crystalline particles are those which have been subjected to a drying process in which not only surface water has been evaporated, but also water of crystallisation has been removed from the crystalline particles.

In the context of the present invention, ‘dried carbohydrate preparation’ is understood to mean a carbohydrate preparation which has a reduced water content of at most 6.0, in particular at most 5.0, in particular at most 1.9 wt.-%, in particular 0.01 to 0.7 wt.-% (based on the total weight of the dried carbohydrate preparation), compared with the water-containing crystalline starting preparation. Preferably, the dried carbohydrate preparation has no syrup film-coated crystalline particles, in particular any water content still present is the water of crystallisation alone.

In the context of the present invention, ‘carbohydrate particle’ is understood to mean at least one carbohydrate which is present in a solid, particulate state. Preferably, the carbohydrate particles are present as a powder. In particular, the carbohydrate particles are present in crystalline form.

In the context of the present invention, ‘semi-crystalline’ is understood to mean a morphological structure of a substance in which the substance has both ordered, crystalline areas and disordered, amorphous areas.

In the context of the present invention, ‘amorphous’ is understood to mean a morphological structure of a compound in which the building blocks, in particular molecules or atoms, are not arranged in a long-range ordered structure. Preferably, the building blocks, in particular molecules, of an amorphous substance form an irregular pattern and have a short-range order. Preferably, amorphous substance have a short-range order and no long-range order.

In the context of the present invention, ‘long-range order’ is understood to mean a regular and periodic arrangement of building blocks, in particular molecules or atoms, in substance beyond their neighbouring building blocks, in particular neighbouring molecules or neighbouring atoms. Accordingly, the exact position of a few building blocks, in particular molecules or atoms, can advantageously be used to determine the position of all building blocks, in particular molecules or atoms, in a crystalline compound. According to the invention, crystalline compounds preferably have at least partially a long-range order.

In the context of the present invention, ‘short-range order’ is understood to mean a regular grouping of building blocks, in particular molecules or atoms, only in the vicinity of a reference building block, in particular reference molecule or reference atom. Preferably, the short-range order refers to the nearest neighbour.

In the context of the present invention, ‘fully crystalline’ is understood to mean a morphological structure of a compound in which the building blocks, in particular molecules or atoms, are regularly arranged in a crystal structure. Preferably, fully crystalline substances have both short-and long-range order.

In the context of the present invention, ‘powdery’ is understood to mean a solid, particulate state of a substance.

In the context of the present invention, ‘angle of repose’ is understood to mean the angle between the surface of a powder cone and its base. Preferably, the angle of repose is calculated from the height of the powder cone h, the radius of the cone r and tan α according to the formula (1): tan α=h/r.

In the context of the present invention, the term ‘free-flowability’ refers to the extent of free mobility of powders or agglomerates. Preferably, the free-flowability is determined, for example, using measuring funnels or free-flowability testers, wherein the free-flow time is measured at a given mass or volume.

In particular, the ‘free-flowability’ is measured according to EUROPEAN PHARMACOPOEIA 10.0 Method 2.9.16. Flowability.

In the context of the present invention, the term ‘stickiness’ is understood to mean the property of a solid carbohydrate preparation measured by means of the stickiness test procedure according to the method section of the Examples. The force can be evaluated as a measure of the adhesive effect of a syrup film on the particle surfaces and thus the strength of the liquid bridges between the particles.

In the context of the present invention, the term ‘caking’ is understood to mean an at least partial or complete solidification of an originally free-flowing substance that occurs over the storage time. Preferably, the solidification can be visually perceived by clump formation. In the context of the present invention, the caking property is determined by means of the test procedure according to the method section of the examples.

In the context of the present invention, the term ‘carbohydrate’ (hereinafter also referred to as saccharide) is understood to mean a sugar and/or a sugar alcohol in monomeric, dimeric or polymeric form. Preferably, ‘sugar’ is understood to mean a mono- or disaccharide sugar, in particular glucose, fructose, isomaltulose, trehalulose and/or sucrose.

In the context of the present invention, ‘sugar alcohol’ is understood to mean a mono- or disaccharide alcohol, in particular isomalt, mannitol and sorbitol.

Where a value for a pressure is given in the context of the present invention, this is to be understood as an absolute pressure and not as a pressure relative to atmospheric pressure. A vacuum is a pressure which is lower than the prevailing atmospheric pressure (1 bar). In particular, a vacuum is understood to be a negative pressure. An excess pressure is a pressure which is higher than the prevailing atmospheric pressure (1 bar).

In the context of the present invention, ‘invert sugar syrup’ is understood to mean an aqueous solution of sucrose partially inverted by hydrolysis with a defined dry matter content and a defined proportion of invert sugar in the dry matter. Invert sugar syrup has in particular a dry matter content of 60 to 70, in particular 65±0.5% (refractometric), an invert sugar content (i.e. a summarily content of glucose and fructose) of 95-99% (HPLC), a glucose content of 47-50% (HPLC), a fructose content of 47-50% (HPLC) and a sucrose content of 1-5% (HPLC).

In the context of the present invention, ‘caramel sugar syrup’ is understood to mean a dark brown aqueous solution of carbohydrates and caramel substances, which is prepared on the basis of sugar by controlled exposure to heat. Caramel sugar syrup preferably has a dry matter content of 70 to 80, in particular 75±0.5% (refractometric), a glucose content of 5-8% (HPLC), a fructose content of 1-4% (HPLC) and a sucrose content of 0-3% (HPLC).

In the context of the present invention, ‘cane sugar syrup’ is understood to mean a sucrose syrup from the sugar cane, which preferably has a dry matter content of 70 to 80, in particular 75±0.5% (refractometric), a glucose content of 32-38% (HPLC), a fructose content of 25-30% (HPLC) and a sucrose content (sucrose from sugar cane) of 10-15% (HPLC).

In the context of the present invention, the term ‘substance X in form of Y’ is understood to mean that the substance X in question is present as a constituent of a composition Y, i.e. X is present together with other, unspecified constituents of Y.

In the context of the present invention, the term ‘minor components’ is understood to mean all substances present in a carbohydrate preparation which are not mono- or disaccharides selected from the group consisting of glucose, fructose, isomaltulose, trehalulose, isomalt and sucrose.

In the context of the present invention, ‘individual minor components’ are individual substances which in their entirety constitute the minor components, wherein these individual substances, for example isomaltose, glycerol, glucopyranosylidite, isomelecitose, are each individual substances belonging to the substance groups of monosaccharides, disaccharides, deoxydisaccharide alcohols, trisaccharides, glucosylglycerols, glucosyltetritols, glucosylpentitols, trisaccharide alcohols, glucosylated disaccharide alcohols or hydrogenated oligomers.

In the context of the present invention, ‘isomalt’ is understood to mean a mixture of 6-O-α-D-glucopyranosyl-D-sorbitol (1,6-GPS) and 1-O-α-D-glucopyranosyl-D-mannitol (1,1-GPM) and optionally 1-O-α-D-glucopyranosyl-D-sorbitol (1,1-GPS), in particular isomalt GS or isomalt ST.

In the context of the present invention, ‘Isomalt GS’ is understood to mean a mixture of 72 to 78 wt.-%, preferably 75 wt.-%, 1,6-GPS and 22 to 28 wt.-%, in particular 25 wt.-%, 1,1-GPM (each based on dry matter of the isomalt). In particular, the isomalt GS has a 1,1-GPM (1-O-alpha-D-glucopyranosyl-D-mannitol) content of 20.0 to 30.0 wt.-% and a 1,6-GPS (6-O-alpha-D-glucopyranosyl-D-sorbitol) content of 70.0 to 80.0 wt.-% (each based on total dry mass of the isomalt).

In the context of the present invention, ‘isomalt ST’ is understood to mean a mixture of 54 to 47 wt.-% 1,6-GPS and 46 to 53 wt.-% 1,1-GPM (each based on the dry weight of the isomalt). In particular, the isomalt ST has a 1,1-GPM (1-O-alpha-D-glucopyranosyl-D-mannitol) content of 45.0 to 50.0 wt.-% and a 1,6-GPS (6-O-alpha-D-glucopyranosyl-D-sorbitol) content of 50 to 55 wt.-% (each based on the total dry mass of the isomalt).

In the context of the present invention, the term ‘isomalt’ or ‘hydrogenated isomaltulose’ is preferably understood to mean a mixture consisting of or comprising 1,1-GPM and 1,6-GPS, in particular a mixture consisting of or comprising 35 to 61 wt.-% of 1,1-GPM and 65 to 39 wt.-% of 1,6-GPS, in particular an equimolar or near-equimolar mixture consisting of or comprising 1,1-GPM and 1,6-GPS (each based on dry matter of the isomalt).

Accordingly, isomalt can also be understood to mean mixtures consisting of or comprising 1,1-GPM and 1,6-GPS which do not have an equimolar ratio of 1,1-GPM to 1,6-GPS, but in which a higher 1,1-GPM content than 1,6-GPS content or a higher 1,6-GPS content than 1,1-GPM content is present.

In a particularly preferred embodiment, the isomalt has no other components in addition to the two components 1,1-GPM and 1,6-GPS.

In a particularly preferred embodiment, the isomalt has, in addition to the two components 1,1-GPM and 1,6-GPS, one or more further components, for example mannitol, sorbitol, sucrose, 1,1-GPS (1-O-α-D-glucopyranosyl-D-sorbitol), glycosylglycitols, deoxy-disaccharide alcohols, GPI (glucopyranosyl-iditol), isomaltose, isomaltulose, isomelecitose, hydrogenated or non-hydrogenated oligosaccharides, in particular hydrogenated or non-hydrogenated trisaccharides, or/and other substances.

In the context of the present invention, an ‘isomaltulose- and trehalulose-containing mixture’ is understood to mean a mixture comprising isomaltulose and trehalulose, in particular obtained from an enzymatic conversion of sucrose by means of a sucrose isomerase to give a sucrose isomer mixture containing isomaltulose and trehalulose, and, optionally, one or more further substances selected from the group consisting of sucrose, fructose, glucose, turanose, leucrose, isomaltose, raffinose, isomelecitose, 6-gluc isomalt and 1-gluc isomalt.

In the context of the present invention, the solubility of a carbohydrate is determined at 20° C. in water, in particular distilled water.

In the context of the present invention, a ‘poorly-soluble carbohydrate’ is understood to be a carbohydrate which has a solubility in pure water, in particular distilled water, at a temperature of 20° C. of at most 53 g/100 g (g dry matter in 100 g solution). A poorly-soluble carbohydrate is in particular isomaltulose, glucose, mannitol and 1,1-GPM.

In the context of the present invention, a ‘well-soluble carbohydrate’ is understood to be a carbohydrate which has a solubility in pure water, in particular distilled water, at a temperature of 20° C. of more than 53 g/100 g, in particular at least 66.7 g/100 g, in particular of at least 68.7 g/100 g, in particular of at least 70.0 g/100 g, in particular of at least 78.9 g/100 g (each g of dry matter in 100 g of solution). A well-soluble carbohydrate is in particular sucrose, 1,6-GPS, fructose, trehalulose and sorbitol.

In the context of the present invention, the diameter of a carbohydrate particle is determined by air jet sieve analysis using a sieve with a mesh size of 100 μm.

The quantitative proportion of particles with a diameter less than or equal to 100 μm is calculated from the proportion passing through the 100 μm mesh sieve in the air jet sieve analysis, divided by the weighed weight.

In the context of the present invention, sucrose-containing starting preparations from sugar cane or sugar beet, although similar, and even if they are purified or highly purified sucrose-containing starting preparations with sucrose contents of more than 99.8 wt.-%, are nevertheless different. In particular, these preparations are different in terms of their flavour profile, their application properties, for example their solubility, their thermal behaviour, the plant-specific impurities regularly present in them and their sensory properties (Lu et al., Journal of Food Engineering (2017), 214, 193 to 208). This is particularly because sugar cane, as a C4 plant belonging to the sweet grass family, has physiological and morphological differences to the C3 plant sugar beet, which belongs to the foxtail family. In particular, it is known that preparations of sucrose molecules originating from C4 plants differ from sucrose from C3 plants, for example sugar beet, due to a metabolic isotope discrimination caused by a different ¹³C/¹²C isotope ratio (Martin et al., Journal of Science of Food and Agriculture, (1991), 56, 419-435). In particular, sucrose-containing starting preparations from C4 plants can therefore be distinguished from those from C3 plants by ¹²C and ¹³C isotope determination, since the ¹³C/¹²C ratio is lower in C3 plants than in C4 plants due to isotope discrimination.

In particular, Averill et al. (Journal of Thermal Analysis and Calorimetry, (2019), 127, 513 to 538) disclose that crystalline sucrose from sugar cane differs from that from beet sugar even at high sucrose purity above 99.8 wt.-% in their thermal behaviour and that in some cases sucrose preparations recovered from sugar cane are characterised by the presence of a small signal detectable in DSC (differential scanning calorimetry) measurements at a temperature below that of the main signal at around 185° C. to 190° C. In contrast, commercial sucrose preparations from sugar beet do not show this melting behaviour, but only have a main signal in the area of 185° C. to 190° C. in DSC measurements (U.S. Pat. No. 8,273,873 B2).

Preferably, the differences in the sucrose preparations originating from these different plant species can be explained in principle by the metabolism of the plant species itself, by the purification process of the sucrose preparation used in each case and by plant-specific impurities which are generally still frequently present in the sucrose preparation obtained, for example syrup residues adhering to sucrose crystals and their solids. A preparation containing sucrose from sugar cane, in particular the entirety of its sucrose molecules, is therefore a different preparation from a preparation containing sucrose from sugar beet, in particular the entirety of its sucrose molecules.

In the context of the present invention, ‘wt.-%’ means ‘weight-%’, ‘GC’ means gas chromatography, ‘HPLC’ means high performance liquid chromatography, ‘Lfd. No.’ means sequence number and “KF” means Karl Fischer method.

In the context of the present invention, the water content is determined tritrimetrically using the Karl Fischer method.

Insofar as ‘presence’, ‘containing’, ‘having’ or ‘content’ of a component is expressly mentioned or implied in the context of the present invention, this means that each component is present, in particular is present in a measurable amount.

If, in connection with the present invention, a ‘presence’, a ‘containing or a ‘having of a component in an amount of 0 [unit], in particular mg/kg, μg/kg or wt.-%, is expressly mentioned or implied, this means that the respective components are not present in a measurable amount, in particular are not present.

Where quantitative data, in particular percentages, of components of a product or a composition are given in the context of the present invention, these add up to 100% of the composition and/or the product together with the other explicitly stated or evident for a person skilled in the art further components of the composition or the product, unless explicitly stated otherwise or evident for a person skilled in the art.

In the context of the present invention, the term ‘at least one’ is understood to mean a quantity expressing a number of 1 or 2 or 3 or 4 or 5 or 6 or 7 or 8 or 9 or 10 and so on. In a particularly preferred embodiment, the term ‘at least one’ may represent exactly the number 1. In a further preferred embodiment, the term ‘at least one’ may also mean 2 or 3 or 4 or 5 or 6 or 7.

The number of decimal places indicated corresponds to the precision of the measurement method used in each case.

If, in the context of the present invention, the first and second decimal places or the second decimal place are/is not specified for a number, it/they is/are to be set as zero.

In the context of the present invention, the term ‘and/or’ is understood to mean that all members of a group which are connected by the term ‘and/or’ are disclosed both alternatively to each other and cumulatively to each other in any combination. This means for the expression ‘A, B and/or C’ that the following disclosure content is to be understood: a) A or B or C or b) (A and B), or c) (A and C), or d) (B and C), or e) (A and B and C).

In the context of the present invention, the terms ‘comprising’ and ‘having’ are understood to mean that, in addition to the elements explicitly covered by these terms, further elements not explicitly mentioned may be added. In the context of the present invention, these terms are also understood to mean that only the explicitly mentioned elements are included and that no further elements are present. In this particular embodiment, the meaning of the terms ‘comprising’ and ‘having’ is synonymous with the term ‘consisting of’. Furthermore, the terms ‘comprising’ and ‘having’ also encompass compositions which, in addition to the explicitly mentioned elements, also contain further elements which are not mentioned but which are of a functional and qualitatively subordinate nature. In this embodiment, the terms ‘comprising’ and ‘having’ are synonymous with the term ‘consisting essentially of’.

Further advantageous embodiments are apparent from the sub-claims.

Without limiting the general idea of the invention, the invention is described in more detail below with reference to examples.

### EXAMPLES

Compositions of the starting materials mentioned in examples 1 and 2

All sucrose is derived from sugar beet, unless otherwise stated (cane sugar syrup).

### Sucrose:

Sucrose: min. 99.7° Z (polarisation)
Invert sugar syrup: <0.04% (enzym.)
Moisture: <0.06% (KF)
   Invert sugar:
Dry matter content: 65±0.5% (refractometric)
Invert sugar content: 95-99% (HPLC)
   Glucose content: 47-50% (HPLC)
   Fructose content: 47-50% (HPLC)
   Sucrose content: 1-5% (HPLC)

### Caramel Sugar Syrup

Dry matter content: 75±0.5% (refractometric)
Glucose content: 5-8% (HPLC)
Fructose content: 1-4% (HPLC)
Sucrose content: 0-3% (HPLC)
   Cane Sugar Syrup (Sucrose from Sugar Cane)
Dry matter content: 75±0.5% (refractometric)
Glucose content: 32-38% (HPLC)
Fructose content: 25-30% (HPLC)
Sucrose content: 10-15% (HPLC)

### Method Section of the Examples—Description of the Test Procedures

### Free-Flowing Test:

The free-flowability was determined according to EUROPEAN PHARMACOPOEIA 10.0 Method 2.9.16. Flowability.

### Stickiness Test:

The stickiness was determined according to the descriptions in: ‘Handbook of Food Science, Endress and Mattes, Technology, and Engineering, 19 Dec. 2005, CRC Press, Volume 3, Chapter 140; Uhlenbrock, Gordian, year 83, pages 148-150 (1983); Food Technology International Europe, 1990, Endress and Dilger, pages 279 to 282; Schilling et al. Eur Food Res Technol (2008) 226:1389-1398; Sirisakulwat et al, International Journal of Food Science and Technology, 20210, 45, 1647-1658; Zedler, ‘Die industrielle Obst-und Gemüseverwertung’ year 68, 12/83, pages 523-527 and Endreß et al, Intern. Zeitschrift für Lebensmittelt-Technologie und-Verfahrenstechnik, 38. year, 1987, Heft 5.

The breaking strength of the gels is assessed according to the tensile force to which a standardised, so-called tearing figure inserted into the gel must be subjected in order to break the gel. ‘A test cup and inserts for test cups=tearing figure are used for this test. A tearing figure is placed in a standardised plastic cup.

The SAP article numbers are Herbstreith Pectinometer Mark IV—901172, Test Cup—900054, Tear Figures—901173.

100 g of the sample to be analysed are placed in the HPE cup. The sample is slightly compacted by lightly tamping the beaker 3 times and left to stand for one hour. The tear figure is then pulled out of the sample using a texture analyser (Winopal) and the required force is determined.

The force can be used as a measure of the strength of the material and thus the strength of the interactions between the crystals.

### Caking Test:

Three metal crucibles are filled ⅔ full for each sample. A plastic film is then placed on the surface of the sample and the surface is weighted down with a stamp (7000 Pa weight). The samples are stored in a climatic cabinet at 25° C. and 65% relative humidity for one week. After storage, the stamp and plastic film are carefully removed and the sample is measured.

To measure the puncture force, a metal needle of a defined geometry is dipped into the surface of the sample and the force is recorded over the path. This is done five times per sample at each untouched point. The force required for dipping-in is an indicator of the compaction or stickiness of the sample.

The measuring device used was the AT-XT Plus Extended Height from Stable Micro Systems.

Example 1: Process according to the invention for preparing a dried and conditioned carbohydrate preparation (samples 2 to 4) and preparation of a comparative sample without the addition of carbohydrate particles (sample 1):

In the following, the preparation is described by way of example using the weights of the sample with the trial number 2, Table 1. The following explanations apply accordingly to the samples according to the invention with the trial numbers 3 to 4, Table 1. Sample 1 represents a comparative sample without the addition of fine crystalline sucrose.

To prepare a water-containing crystalline carbohydrate starting preparation, 922.0 g of sucrose in the form of granulated sugar (Südzucker EU2 quality grain size M) is placed in a coating kettle function as a drying reactor. In a heated and stirred vessel, 60 g of caramel sugar syrup (component B) and 18 g of cane sugar syrup (component C) are mixed and heated to approximately (abbreviated as approx. in the following) 60° C. The heated syrup mixture obtained is slowly added to the sucrose in a rotating coating kettle (process step a1)). The resulting mixture is homogenised for about 5 minutes and a water-containing crystalline carbohydrate starting preparation according to process step a) of the present invention is obtained, wherein the crystalline sucrose particles are coated with a syrup film. For samples 2 to 4, the amount of fine crystalline sucrose (at least 80 wt.-% smaller than 100 μm and at least 70 wt.-% smaller than 32 μm) indicated in Table 4 is metered into the water-containing crystalline carbohydrate starting preparation with a temperature of 21 to 22° C. via a swivelling channel at a temperature of 20° C. under atmospheric pressure, at approx. 1000 mbar according to process step b) of the present invention. In trial 1, metering-in of fine crystalline sucrose is omitted. The mixture with a temperature of 20 to 21° C. is homogenised for 5 minutes under atmospheric pressure at approx. 1000 mbar according to process step c) of the present invention until the carbohydrate particles are visually recognizably distributed. Compositions of the dried carbohydrate preparation, listed in Tables 5 and 6, are obtained according to the process of the invention. For conditioning according to process step d) of the present invention, ambient air preheated to about 90° C. is passed over the dried carbohydrate preparation by means of a blower. The dried carbohydrate preparation reaches a temperature of about 50° C. A dried and conditioned carbohydrate preparation is obtained (Table 7) when the mixture has become particularly easily free-flowing and no longer has any clumps. The heating of the blower is switched off and ambient air at room temperature is passed over the dried and conditioned carbohydrate preparation until it has reached a temperature of below 35° C.

Comparative sample 1 serves as a control. As no fine crystalline sucrose is added, no free-flowing product is obtained during the conditioning experiment with warm air, but a viscous and very sticky mass. No further trials or measurements can be carried out with this product (n.a., not measurable, see Tables 7 and 8).

The characteristics of the dried and conditioned carbohydrate preparations obtained by the process according to the invention are shown in Tables 1 to 7 below and the results of trials on the free-flowability and angle of repose of each conditioned carbohydrate preparation are shown in Table 8. The conditioned carbohydrate preparations according to the invention show a particularly good angle of repose and a particularly good free-flowability.

TABLE 1
Quantities of starting substances for preparing the
crystalline water-containing sucrose preparation

| | | | Component A | Component B | Component C |
|---|---|---|---|---|---|
| Trial | Lfd. | Sucrose | Invert sugar | Caramel sugar | Cane sugar |
| number | No. | [g] | syrup [g] | syrup [g] | syrup [g] |
| 1 | 202111129 | 922.0 | | 60.0 | 18.0 |
| 2 | 202111133 | 922.0 | — | 60.0 | 18.0 |
| 3 | 202111134 | 922.0 | — | 60.0 | 18.0 |
| 4 | 202111135 | 922.0 | — | 60.0 | 18.0 |

TABLE 2
Composition of the crystalline water-containing sucrose preparation

| Trial | | Sucrose | Glucose | Fructose | Remainder | Water |
|---|---|---|---|---|---|---|
| number | Lfd. No. | [%] | [%] | [%] | [%] | [%] |
| 1 | 202111129 | 92.39 | 0.76 | 0.49 | 4.45 | 1.91 |
| 2 | 202111133 | 92.39 | 0.76 | 0.49 | 4.45 | 1.91 |
| 3 | 202111134 | 92.39 | 0.76 | 0.49 | 4.45 | 1.91 |
| 4 | 202111135 | 92.39 | 0.76 | 0.49 | 4.45 | 1.91 |

TABLE 3
Composition of the dry matter of the crystalline
water-containing sucrose preparation

| Trial | Lfd. | Sucrose | Glucose | Fructose | Remainder |
|---|---|---|---|---|---|
| number | No. | [%] | [%] | [%] | [%] |
| 1 | 202111129 | 94.19 | 0.77 | 0.50 | 4.53 |
| 2 | 202111133 | 94.19 | 0.77 | 0.50 | 4.53 |
| 3 | 202111134 | 94.19 | 0.77 | 0.50 | 4.53 |
| 4 | 202111135 | 94.19 | 0.77 | 0.50 | 4.53 |

TABLE 4
Quantities of starting substances used to prepare the dried
carbohydrate preparations and comparative sample 1

| | | | Component A | Component B | Component C | Fine |
|---|---|---|---|---|---|---|
| Trial | | Sucrose | Invert sugar | Caramel sugar | Cane sugar | cristalline |
| number | Lfd. No. | [g] | syrup [g] | syrup [g] | syrup [g] | sucrose [g] |
| 1 | 202111129 | 922.0 | | 60.0 | 18.0 | — |
| 2 | 202111133 | 922.0 | — | 60.0 | 18.0 | 20.0 |
| 3 | 202111134 | 922.0 | — | 60.0 | 18.0 | 50.0 |
| 4 | 202111135 | 922.0 | — | 60.0 | 18.0 | 100.0 |

TABLE 5
Composition of the dried carbohydrate preparations and of comparative sample 1

| Trial | | Sucrose | Glucose | Fructose | Remainder | Water |
|---|---|---|---|---|---|---|
| number | Lfd. No. | [%] | [%] | [%] | [%] | [%] |
| 1 | 202111129 | 92.39 | 0.76 | 0.49 | 4.45 | 1.91 |
| 2 | 202111133 | 92.54 | 0.74 | 0.48 | 4.36 | 1.87 |
| 3 | 202111134 | 92.76 | 0.72 | 0.47 | 4.24 | 1.82 |
| 4 | 202111135 | 93.08 | 0.69 | 0.44 | 4.04 | 1.74 |

TABLE 6
Composition of the total dry mass of the dried carbohydrate
preparations and of comparative sample 1

| Trial | Lfd. | Sucrose | Glucose | Fructose | Remainder |
|---|---|---|---|---|---|
| number | No. | [%] | [%] | [%] | [%] |
| 1 | 202111129 | 94.19 | 0.77 | 0.50 | 4.53 |
| 2 | 202111133 | 94.31 | 0.76 | 0.49 | 4.44 |
| 3 | 202111134 | 94.47 | 0.74 | 0.47 | 4.31 |
| 4 | 202111135 | 94.73 | 0.70 | 0.45 | 4.11 |

TABLE 7
Composition of the dried and conditioned carbohydrate
preparations and of comparative sample 1

| Trial | | Sucrose | Glucose | Fructose | Remainder | Water |
|---|---|---|---|---|---|---|
| number | Lfd. No. | [%] | [%] | [%] | [%] | [%] |
| 1 | 202111129 | n.a. | n.a. | n.a. | n.a. | n.a. |
| 2 | 202111133 | 94.05 | 0.76 | 0.49 | 4.43 | 0.28 |
| 3 | 202111134 | 94.20 | 0.73 | 0.47 | 4.30 | 0.29 |
| 4 | 202111135 | 94.33 | 0.70 | 0.45 | 4.10 | 0.42 |

n.a.: not measurable

TABLE 8
Free-flowability (abbreviated as f.-flow.) and angle of repose (abbreviated as ang. rep.)
of the dried and conditioned carbohydrate preparations and of comparative sample 1

| | | ang. | f.-flow. | f.-flow. | f.-flow. | f.-flow. | f.-flow. | f.-flow. |
|---|---|---|---|---|---|---|---|---|
| Versuchs- | | rep. | 6 mm | 8 mm | 10 mm | 11.3 mm | 15 mm | 25 mm |
| nummer | Lfd. No. | [°] | [s/100 g] | [s/100 g] | [s/100 g] | [s/100 g] | [s/100 g] | [s/100 g] |
| 1 | 202111129 | n.a. | n.a. | n.a. | n.a. | n.a. | n.a | n.a. |
| 2 | 202111133 | 33.7 | 32.6 | 14.7 | 7.7 | 4.5 | 2 | 0.1 |
| 3 | 202111134 | 36.4 | n.a. | 17.4 | 9.3 | 5.3 | 2.4 | 0.2 |
| 4 | 202111135 | 38.5 | n.a. | 17.4 | 9.7 | 6.1 | 2.7 | 0.3 |

n.a.: not measurable

Example 2: Comparison of carbohydrate preparations obtained according to the invention (brown sugar type A) (samples 2 to 9) and a comparative sample without the addition of carbohydrate particles (sample 1).

The preparation of the dried carbohydrate preparations obtained according to the invention and of the comparative sample 1 was carried out analogously to Example 1. Tables 9 to 15 show the characteristics of the comparative sample 1 (without addition of fine crystalline sucrose, i.e. without carrying out process steps b) and c)), of dried products according to the invention (samples 2 to 5) and of dried and conditioned products according to the invention (samples 6 to 9).

The samples were characterised and compared, as far as possible, on the basis of the water content (KF method) in total (total water) and the water content on the surface (KF method) (surface water), the angle of repose and the free-flowability. The icing sugar used (fine crystalline sucrose) corresponds to the crystalline sucrose according to Example 1.

The results are summarised in Table 16. It can be seen here that dried brown sugar type A samples (samples 2 to 5) show a significantly increased free-flowability by increasing the amount of fine crystalline sucrose added from 2 wt.-% to up to 20 wt.-% (based on the starting weight of brown sugar type A sample) and in comparison to the comparative example (sample 1.0 wt.-% carbohydrate particles in the form of fine crystalline sucrose). For example, Table 16 in particular shows that the measured time required is significantly reduced when samples according to the invention are used in comparison with sample 1, in particular with a discharge opening size of 15 mm. The comparative sample 1 shows no measurable free-flowability at all with outlet opening sizes smaller than 15 mm (n.a. in Table 16), unlike the samples according to the invention, which have good free-flowability even with smaller openings.

It is shown that as the amount of fine crystalline carbohydrate particles added increases, the time required decreases from 2.3 s (comparative example, sample 1.0 wt.-% finely crystalline carbohydrate particles) to 0.2 s (sample 5, 20 wt.-% fine crystalline carbohydrate particles) (free-flowability 25 mm). Furthermore, the dried samples show a decreasing total water content as well as a decreasing water content at the surface of the samples with increasing addition of the fine crystalline carbohydrate particles.

In addition to the process steps b) and c) according to the invention, samples 6 to 9 were subjected to a subsequent conditioning step d), as described in example 1. The dried and conditioned preparations obtained had almost the same behaviour as samples 2 to 5, wherein samples 6 to 9 have an even better free-flowability and lower total water content and water content on the surface of the samples compared to the dried samples 2 to 5 with the same amount of fine crystalline carbohydrate particles added and in particular with low addition amounts of fine crystalline carbohydrate particles of 2% and 5% (samples 6 and 7 compared to samples 2 and 3). The water content in the dried and conditioned samples from 10% addition of fine crystalline carbohydrate particles (samples 8 and 9) does not decrease further. For dried and conditioned samples, it can also be seen that the angle of repose decreases as the amount of fine crystalline carbohydrate particles increases, which also indicates increased free-flowability. In this example, the control sample (sample 1) was also subjected to the conditioning step. The result was a very doughy, very sticky product with unmeasurable free-flowability.

TABLE 9
Quantities of starting substances for preparing the
crystalline water-containing sucrose preparation

| Trial | Llf. | Sucrose | Invert sugar | Caramel sugar |
|---|---|---|---|---|
| no. | No. | (g) | syrup (g) | syrup (g) |
| 1 | 202108480 | 979.8 | 14.1 | 6.1 |
| 2 | 202108481 | 979.8 | 14.1 | 6.1 |
| 3 | 202108482 | 979.8 | 14.1 | 6.1 |
| 4 | 202108483 | 979.8 | 14.1 | 6.1 |
| 5 | 202108484 | 979.8 | 14.1 | 6.1 |
| 6 | 202108485 | 979.8 | 14.1 | 6.1 |
| 7 | 202108486 | 979.8 | 14.1 | 6.1 |
| 8 | 202108487 | 979.8 | 14.1 | 6.1 |
| 9 | 202108488 | 979.8 | 14.1 | 6.1 |

TABLE 10
Composition of the crystalline water-containing
sucrose preparation according to Table 9

| Trial | | Sucrose | Glucose | Fructose | Remainder | Water |
|---|---|---|---|---|---|---|
| no. | Lfd. No. | in % | in % | in % | in % | % |
| 1 | 202108480 | 97.99 | 0.49 | 0.45 | 0.44 | 0.64 |
| 2 | 202108481 | 97.99 | 0.49 | 0.45 | 0.44 | 0.64 |
| 3 | 202108482 | 97.99 | 0.49 | 0.45 | 0.44 | 0.64 |
| 4 | 202108483 | 97.99 | 0.49 | 0.45 | 0.44 | 0.64 |
| 5 | 202108484 | 97.99 | 0.49 | 0.45 | 0.44 | 0.64 |
| 6 | 202108485 | 97.99 | 0.49 | 0.45 | 0.44 | 0.64 |
| 7 | 202108486 | 97.99 | 0.49 | 0.45 | 0.44 | 0.64 |
| 8 | 202108487 | 97.99 | 0.49 | 0.45 | 0.44 | 0.64 |
| 9 | 202108488 | 97.99 | 0.49 | 0.45 | 0.44 | 0.64 |

TABLE 11
Composition of the total dry mass of the water-containing
carbohydrate preparations according to Table 10

| Trial | Lfd. | Sucrose | Glucose | Fructose | Remainder |
|---|---|---|---|---|---|
| no. | No. | in % | in % | in % | in % |
| 1 | 202108480 | 98.62 | 0.49 | 0.45 | 0.44 |
| 2 | 202108481 | 98.62 | 0.49 | 0.45 | 0.44 |
| 3 | 202108482 | 98.62 | 0.49 | 0.45 | 0.44 |
| 4 | 202108483 | 98.62 | 0.49 | 0.45 | 0.44 |
| 5 | 202108484 | 98.62 | 0.49 | 0.45 | 0.44 |
| 6 | 202108485 | 98.62 | 0.49 | 0.45 | 0.44 |
| 7 | 202108486 | 98.62 | 0.49 | 0.45 | 0.44 |
| 8 | 202108487 | 98.62 | 0.49 | 0.45 | 0.44 |
| 9 | 202108488 | 98.62 | 0.49 | 0.45 | 0.44 |

TABLE 12
Quantities of starting substances used to prepare the dried
crystalline carbohydrate preparation and comparative sample 1

| Trial | Lfd. | Sucrose | Invert sugar | Caramel sugar | Fine crystalline |
|---|---|---|---|---|---|
| no. | No. | (g) | syrup (g) | syrup (g) | sucrose (g) |
| 1 | 202108480 | 979.8 | 14.1 | 6.1 | — |
| 2 | 202108481 | 979.8 | 14.1 | 6.1 | 20.0 |
| 3 | 202108482 | 979.8 | 14.1 | 6.1 | 50.0 |
| 4 | 202108483 | 979.8 | 14.1 | 6.1 | 100.0 |
| 5 | 202108484 | 979.8 | 14.1 | 6.1 | 200.0 |
| 6 | 202108485 | 979.8 | 14.1 | 6.1 | 20.0 |
| 7 | 202108486 | 979.8 | 14.1 | 6.1 | 50.0 |
| 8 | 202108487 | 979.8 | 14.1 | 6.1 | 100.0 |
| 9 | 202108488 | 979.8 | 14.1 | 6.1 | 200.0 |

TABLE 13
Composition of the dried carbohydrate preparations no. 2 to
9 (before conditioning according to process step d) for samples
6 to 9) and of comparative sample 1 according to Table 12

| Trial | | Sucrose | Glucose | Fructose | Remainder | Water |
|---|---|---|---|---|---|---|
| no. | Lfd. No. | in % | in % | in % | in % | % |
| 1 | 202108480 | 97.99 | 0.49 | 0.45 | 0.44 | 0.64 |
| 2 | 202108481 | 98.03 | 0.48 | 0.44 | 0.43 | 0.63 |
| 3 | 202108482 | 98.08 | 0.46 | 0.43 | 0.42 | 0.61 |
| 4 | 202108483 | 98.17 | 0.44 | 0.41 | 0.40 | 0.58 |
| 5 | 202108484 | 98.32 | 0.41 | 0.37 | 0.36 | 0.53 |
| 6 | 202108485 | 98.03 | 0.48 | 0.44 | 0.43 | 0.63 |
| 7 | 202108486 | 98.08 | 0.46 | 0.43 | 0.42 | 061 |
| 8 | 202108487 | 98.17 | 0.44 | 0.41 | 0.40 | 0.58 |
| 9 | 202108488 | 98.32 | 0.41 | 0.37 | 0.36 | 0..53 |

TABLE 14
Composition of the total dry mass of the carbohydrate
preparations according to Table 13

| Trial | Lfd. | Sucrose | Glucose | Fructose | Remainder |
|---|---|---|---|---|---|
| no. | No. | in % | in % | in % | in % |
| 1 | 202108480 | 98.62 | 0.49 | 0.45 | 0.44 |
| 2 | 202108481 | 98.65 | 0.48 | 0.44 | 0.43 |
| 3 | 202108482 | 98.69 | 0.47 | 0.43 | 0.42 |
| 4 | 202108483 | 98.75 | 0.45 | 0.41 | 0.40 |
| 5 | 202108484 | 98.85 | 0.41 | 0.37 | 0.37 |
| 6 | 202108485 | 98.65 | 0.48 | 0.44 | 0.43 |
| 7 | 202108486 | 98.69 | 0.47 | 0.43 | 0.42 |
| 8 | 202108487 | 98.75 | 0.45 | 0.41 | 0.40 |
| 9 | 202108488 | 98.85 | 0.41 | 0.37 | 0.37 |

TABLE 15
Composition of the dried (No. 2 to 5) and dried and conditioned (No.
6 to 9) carbohydrate preparations and of comparative sample 1

| Trial | | Sucrose | Glucose | Fructose | Remainder | Water |
|---|---|---|---|---|---|---|
| no. | Lfd. No. | in % | in % | in % | in % | % |
| 1 | 202108480 | 97.99 | 0.49 | 0.45 | 0.44 | 0.64 |
| 2 | 202108481 | 98.03 | 0.48 | 0.44 | 0.43 | 0.63 |
| 3 | 202108482 | 98.08 | 0.46 | 0.43 | 0.42 | 0.61 |
| 4 | 202108483 | 98.17 | 0.44 | 0.41 | 0.40 | 0.58 |
| 5 | 202108484 | 98.32 | 0.41 | 0.37 | 0.36 | 0.53 |
| 6 | 202108485 | 98.52 | 0.48 | 0.44 | 0.43 | 0.13 |
| 7 | 202108486 | 98.42 | 0.47 | 0.43 | 0.42 | 0.27 |
| 8 | 202108487 | 98.63 | 0.45 | 0.41 | 0.40 | 0.12 |
| 9 | 202108488 | 98.72 | 0.41 | 0.37 | 0.36 | 0.13 |

TABLE 16
Angle of repose and free-flowability as well as water content and water content on the surface of various brown sugar type A samples
(comparative example without added icing sugar (comparative sample 1) and dried carbohydrate preparations according to the invention
(samples 2 to 5) or dried and conditioned carbohydrate preparations according to the invention (samples 6 to 9))

| | | Fine | | | f.-flow. | f.-flow. | f.-flow. | f.-flow. | f.-flow. | f.-flow. | Water | Water |
|---|---|---|---|---|---|---|---|---|---|---|---|---|
| | | crystalline | | ang. | 6 mm | 8 mm | 10 mm | 11.3 mm | 15 mm | 25 mm | total | surface |
| | | sucrose | | rep. | [s/100 | [s/100 | [s/100 | [s/100 | [s/100 | [s/100 | KF | KF |
| Sample | Lfd. No. | [wt-%] | conditioned | [°] | g] | g] | g] | g] | g] | g] | [g/100 g] | [g/100 g] |
| 1 | 202108480 | 0 | No | n. a. | n. a. | n. a. | n. a. | n. a. | 99.78 | 2.3 | 0.49 | 0.47 |
| 2 | 202108481 | 2 | No | n. a. | n. a. | n. a. | 18.1 | 12.6 | 6.5 | 1.9 | 0.46 | 0.43 |
| 3 | 202108482 | 5 | No | 40.7 | n. a. | 22.5 | 13.2 | 8.1 | 3.9 | 0.6 | 0.44 | 0.42 |
| 4 | 202108483 | 10 | No | 38.1 | 34.2 | 15.5 | 8.2 | 5.2 | 2.3 | 0.2 | 0.37 | 0.34 |
| 5 | 202108484 | 20 | No | 41.9 | 39.1 | 15.7 | 8.3 | 5.3 | 2.2 | 0.2 | 0.33 | 0.31 |
| 6 | 202108485 | 2 | Yes | n. a. | 28.5 | 14.8 | 8.2 | 6.1 | 4.6 | 2.5 | 0.13 | 0.067 |
| 7 | 202108486 | 5 | Yes | 40.8 | 36.4 | 15.1 | 8.3 | 5.4 | 3.8 | 1.3 | 0.27 | 0.084 |
| 8 | 202108487 | 10 | Yes | 35.1 | 36.2 | 15.0 | 8.0 | 5.3 | 2.1 | 0.1 | 0.12 | 0.054 |
| 9 | 202108488 | 20 | Yes | 36.6 | 30.6 | 12.6 | 6.6 | 4.1 | 1.7 | 0.0 | 0.13 | 0.076 |

Example 3 Process according to the invention for preparing a dried and conditioned isomaltulose- and trehalulose-containing composition and preparation of a comparative sample without addition of carbohydrate particles

In the following, the preparation of a dried isomaltulose- and trehalulose-containing preparation according to the invention from a water-containing crystalline isomaltulose- and trehalulose-containing starting preparation is described and its properties with regard to its free-flowability and stickiness are shown.

Firstly, the starting material of the present invention, i.e. the water-containing crystalline carbohydrate starting preparation, is prepared from a solid crystalline isomaltulose- and trehalulose-containing preparation (hereinafter also referred to as ‘crystalline preparation’) and an aqueous syrup of an isomaltulose- and trehalulose-containing preparation to be applied thereto, wherein carbohydrate particles coated with a syrup film are formed. This preparation step, which provides the starting material for the process according to the invention, serves to provide a starting product for the present examples in a standardised manner and leads to a product as it is obtained in industrial production and is further processed in subsequent industrial drying steps. These syrup film-coated particles are then dried in according to the invention by carrying out process steps b) and c) (addition of powder) and then, optionally, conditioned.

TABLE 17
Analysis of the solid crystalline isomaltulose and trehalulose-containing preparation -
(composition of the sugar spectrum by GC (gas chromatography) (GC-DB5-OTMS)

| | | Water | | | | | |
|---|---|---|---|---|---|---|---|
| | f.-flow. | content | Fructose | Glucose | sucrose | Trehalu. | Isomaltulose |
| | 15 mm | total | GC- | GC- | GC- | GC- | GC- |
| | nozzle | KF | DB5- | DB5- | DB5- | DB5- | DB5- |
| Sample | chronom. | titrim. | OTMS | OTMS | OTMS | OTMS | OTMS |
| designation | s/100 g | g/100 g | g/100 g | g/100 g | g/100 g | g/100 g | g/100 g |
| Crystalline | 3.0 | 5.1 | 0.06 | 0.04 | 0.01 | 0.43 | 93.8 |
| preparation | | | | | | | |

Trehalu. means trehalulose

Powder type 1 and type 2 of the solid crystalline isomaltulose and trehalulose-containing preparation had the same composition as the crystalline preparation shown in Table 17 above, but differ from each other in terms of particle size. Powder type 1 has a particle size of at least 90 wt.-% smaller than 0.05 mm. Powder type 2 has a particle size of at least 90 wt.-% smaller than 0.1 mm.

TABLE 18
Analysis of the syrup of a isomaltulose- and trehalulose-containing preparation
(Composition of the sugar spectrum according to GC (HPLC-NH2)

| | | Fructose | Glucose | Sucrose | Trehalu. | Isomaltulose |
|---|---|---|---|---|---|---|
| | DM | HPLC- | HPLC- | HPLC- | HPLC- | HPLC- |
| Sample | ref. | NH2 | NH2 | NH2 | NH2 | NH2 |
| designation | g/100 g | g/100 g | g/100 g | g/100 g | g/100 g | g/100 g |
| Syrup Type A | 39.96 | 1.2 | 0.7 | 0.5 | 3.4 | 33 |

Preparing without Adding Powder (Control):

In a 3 L mixer with a flat stirrer 1 kg of crystalline isomaltulose- and trehalulose-containing preparation was placed. The appropriate amount of aqueous syrup of an isomaltulose- and trehalulose-containing preparation is sprayed slowly, in small portions and at defined intervals onto the crystalline isomaltulose- and trehalulose-containing preparation. This is followed by a mixing time of approximately 5 minutes to distribute the syrup homogeneously. Once the mixing time is complete, the mixture is conditioned with warm air. The temperature of the mass is approx. 35° C.

Carrying Out with the Addition of Powder (According to the Invention):

In a 3 L mixer with a flat stirrer 1 kg of crystalline isomaltulose- and trehalulose-containing preparation was placed. The corresponding amount of aqueous syrup of a isomaltulose- and trehalulose-containing preparation is sprayed slowly, in small portions and at defined intervals onto the crystalline isomaltulose- and trehalulose-containing preparation. This is followed by a mixing time of approximately 5 minutes to distribute the syrup homogeneously. After the addition is complete, a fine-crystalline isomaltulose- and trehalulose-containing preparation (powder) is added over the mass at atmospheric pressure and room temperature of 21 to 22° C. using a metering device. The mixture with the temperature of 20 to 21° C. is homogenised for 5 minutes under atmospheric pressure at about 1000 mbar according to process step c) of the present invention until the carbohydrate particles are optically recognizably distributed.

Compositions of the dried carbohydrate preparation according to the process of the invention are obtained. The mixture is then conditioned with warm air. The temperature of the mass is approximately 35° C.

In a first trial series, the solid crystalline isomaltulose- and trehalulose-containing preparation was treated with 2.0 or 9.6 wt.-% (based on the total weight of the syrup film-coated starting preparation) of an isomaltulose- and trehalulose-containing syrup type A and then coated with 4.6 wt.-% (based on the total weight of the syrup film-coated starting preparation including the weight of the powder), respectively, of an isomaltulose- and trehalulose-containing fine crystalline powder of a type 1 or type 2.

TABLE 19
First test series

| | crystalline | Syrup | Powder | Powder |
|---|---|---|---|---|
| Trial | preparation | type A | type 1 | type 2 |
| no. | g | g | g | g |
| 1 | 1000 | 20 | 0 | 0 |
| 2 | 1000 | 106 | 0 | 0 |
| 3 | 1000 | 106 | 53 | 0 |
| 4 | 1000 | 106 | 0 | 53 |

In a second trial series, the solid crystalline isomaltulose- and trehalulose-containing preparation was treated with 2.0 or 4.8 wt.-% (based on the total weight of the syrup film-coated starting preparation) of type A syrup and then coated with 4.8 wt.-% (based on the total weight of the syrup film-coated starting preparation including the weight of the powder) of the fine crystalline powder type 1 or type 2 respectively.

TABLE 20
Second trial series

| | crystalline | Syrup | Powder | Powder |
|---|---|---|---|---|
| Trial | preparation | type A | type 1 | type 2 |
| no. | g | g | g | g |
| 1 | 1000 | 20 | 0 | 0 |
| 5 | 1000 | 50 | 0 | 0 |
| 6 | 1000 | 50 | 0 | 53 |
| 7 | 1000 | 50 | 53 | 0 |

### Free-Flowability:

Table 21 summarises the results of the first trial series. There is an increase in the free-flowability time measured in a 15 mm nozzle from the crystalline preparation (control) to the two water-containing starting preparations coated with syrup in trials 1 and 2. When the fine crystalline powders types 1 and 2 are used according to the invention in trials 3 and 4, the free-flowability time decreases again.

TABLE 21
Results of the analyses of the products of trial series 1

| | f.-flow. | Water content | |
|---|---|---|---|
| | 15 mm nozzle | total after KF | Trehalulose |
| Trial | chronom. | titrim. | GC-DB5-OTMS |
| No. | s/100 g | g/100 g | g/100 g |
| Crystalline | 3.0 | 5.10 | 0.43 |
| preparation | | | |
| 1 | 4.2 | 5.33 | 0.51 |
| 2 | 4.8 | 5.47 | 0.75 |
| 3 | 2.3 | 5.20 | 0.81 |
| 4 | 2.4 | 5.18 | 0.72 |

Chronom. means chronometric, s/100 g: seconds of flow time per 100 g, tritrim. means titrimetric, g/100 g: grams of water per 100 g.

An analogue picture emerges for trial series 2. Table 22 summarises the results of the second trial series. There is an increase in the free-flowability time from the crystalline preparation (control) to the two water-containing starting preparations coated with syrup in trials 1 and 5. When the fine crystalline powders types 1 and 2 are used according to the invention in tests 5 and 6, the free-flowability time decreases again.

TABLE 22
Results of the analyses of the products of trial series 2

| | f.-flow. | Water content | |
|---|---|---|---|
| | 15 mm nozzle | total after KF | Trehalulose |
| Trial | chronom. | titrim. | GC-DB5-OTMS |
| No. | s/100 g | g/100 g | g/100 g |
| Crystalline | 3.0 | 5.10 | 0.43 |
| preparation | | | |
| 1 | 4.2 | 5.33 | 0.51 |
| 5 | 6.2 | 5.41 | 0.59 |
| 6 | 2.7 | 5.10 | 0.58 |
| 7 | 2.9 | 5.37 | 0.58 |

### Determination of Stickiness:

The determination of stickiness was carried out on three selected samples; the crystalline isomaltulose and trehalulose-containing preparation before syrup treatment (trial 1), the water-containing crystalline isomaltulose and trehalulose-containing starting preparation coated with syrup (trial 2) and the water-containing crystalline isomaltulose and trehalulose-containing starting preparation coated with syrup, which was subsequently treated with a powder of a crystalline isomaltulose- and trehalulose-containing preparation according to the invention and dried (trial 3).

The measured values according to Table 23 show that the crystalline preparation is the least sticky, with a strength value of 84.5 g. Treatment with syrup (trial 2) results in a significantly higher force, indicating the significantly higher stickiness. After treating these samples with microcrystalline powder type 1 according to the invention (trial 3), the stickiness is significantly reduced.

TABLE 23
Stickiness of selected samples

| Trial | Force in g |
|---|---|
| Crystalline | 84.5 |
| preparation | |
| 2 | 132.4 |
| 3 | 109.1 |

### Caking Test:

A caking test was carried out on the samples according to tests 1 to 3 for stickiness. A caking test can be used to determine the tendency of a material to caking when it is compacted. The measured value is determined immediately without further storage and after one week of storage. The caking tendency of a particulate material depends on the stickiness of the individual particles.

The measured values determined from the non-stored samples show practically no significant difference and are therefore not listed. Table 24 shows the values measured after one week. The crystalline preparation (trial 1) shows a particularly low value of 0.26 N. The water-containing starting preparation, which was therefore coated with syrup according to trial 2, shows a very significant increase in the force value to 6.33 N. After treatment with powder in trial 3, the value drops very significantly to a value of 1.50 N.

TABLE 24
Caking of selected samples

| | Force in N |
|---|---|
| Trial | after 1 week |
| crystalline | 0.26 |
| preparation | |
| 2 | 6.33 |
| 3 | 1.50 |

The trials show that it is possible to produce a water-containing isomaltulose- and trehalulose-containing starting preparation by using a fine crystalline isomaltulose- and trehalulose-containing preparation (powder), which has been prepared from a crystalline isomaltulose- and trehalulose-containing preparation by coating with a syrup of an isomaltulose- and trehalulose-containing preparation, improved in terms of free-flowability, in particular stickiness and caking tendency. Surprisingly, there is no caking or clumping, but a significant improvement in free-flowability.

Example 4 Process according to the invention for preparing a dried and conditioned isomalt-containing composition and preparation of a comparative sample without the addition of carbohydrate particles

In the following, the preparation of a dried isomalt-containing preparation according to the invention from a water-containing crystalline isomalt-containing starting preparation is described and its properties with regard to its free-flowability and stickiness are shown.

### Characterisation of the Starting Materials:

Crystalline isomalt type 1 (at least 90% of crystals with a diameter of 0.8-1.5 mm),
Isomalt solution (syrup),
Isomalt powder type 1 (at least 90% of the crystals with a diameter of <0.1 mm) (powder).

To prepare the crystalline water-containing isomalt starting preparation used in process step a), solid crystalline isomalt type 1 (hereinafter also referred to as ‘crystalline preparation’) was first coated with isomalt solution, wherein carbohydrate particles coated with a syrup film are formed. This preparation step, which provides the starting material of the process according to the invention, serves to provide a starting product for the present examples in a standardised manner and leads to a product as it is obtained in industrial production and is further processed in subsequent industrial drying steps. These syrup film-coated particles are then dried according to the invention by carrying out process steps b) and c) (addition of powder) and then, optionally, conditioned.

Trials without Adding Powder (Control):

In a 5 L coating kettle 1 kg of crystalline isomalt is placed. The coating kettle is rotated at approx. 21 rpm. The appropriate amount of isomalt solution is sprayed slowly, in small portions and at defined intervals onto the isomalt. Between spraying, the mixture is dried briefly using warm air and a crystalline water-containing isomalt starting preparation is obtained in which the isomalt crystals are coated with a syrup film. After the addition of the isomalt solution is complete, the mixture is conditioned with warm air for around 10 minutes. The temperature of the mass is approximately 45° C. The mixture is sieved to remove any agglomerates.

Trials with the Addition of Powder (According to the Invention):

In a 5 L coating kettle 1 kg of crystalline isomalt is placed. The coating kettle is rotated at approx. 21 rpm. The appropriate amount of the isomalt solution is sprayed slowly, in small portions and at defined intervals onto the isomalt. Between spraying, the mixture is dried briefly using warm air and a crystalline water-containing isomalt starting preparation is obtained in which the isomalt crystals are coated with a syrup film. After the addition has been completed, the fine-crystalline isomalt component (powder type 1) is added on the mass at atmospheric pressure and room temperature of 21 to 22° C. in an amount of 4.6 wt.-% (based on the total weight of the syrup film-coated starting preparation including the powder component) via a metering device. The mixture with a temperature of 20 to 21° C. is homogenised for 5 minutes under atmospheric pressure at approx. 1000 mbar according to process step c) of the present invention until the carbohydrate particles are visually recognizably distributed. After the addition is complete, the dried isomalt preparation obtained according to the invention is conditioned with warm air for about 10 minutes. The temperature of the mass is between approx. 45° C. The mixture is sieved to remove any agglomerates that may occur.

TABLE 25
Trial overview with formulation quantities

| | Isomalt | Isomalt | Isomalt powder |
|---|---|---|---|
| No. | Type 1 | solution | Type 1 |
| — | [g] | [g] | [g] |
| 1 | 1000 | 50 | — |
| 2 | 1000 | 106 | — |
| 3 | 1000 | 106 | 53 |

TABLE 26
Analyses of the starting materials - Part 1

| | f.-flow. | |
|---|---|---|
| | chronom. | |
| | Ph. Eur. | |
| | 2.9.16 | |
| | s/100 g | |
| Designation | MW | nozzle |
| Isomalt | 7.4 | Measured with |
| Type 1 | | 10 mm nozzle |

TABLE 27
Analyses of the starting materials - Part 2

| | GPM | GPS | Sorbitol |
|---|---|---|---|
| | GC-HT8- | GC-HT8- | GC-HT8- |
| | BOTMS | BOTMS | BOTMS |
| | g/100 gDM | g/100 gDM | g/100 gDM |
| designation | MW | MW | MW |
| Isomalt Type 1 | 47.7 | 51.1 | 0.09 |
| Isomalt powder | 47.7 | 51.7 | 0.11 |
| Type 1 | | | |

TABLE 28
Analyses of the starting materials - Part 3
Product analysis Isomalt syrup

| Parameter | Real-value | unit |
|---|---|---|
| DM (refr.) corr. | 40.7 | % |
| Mannitol | 1.7 | standard-% |
| Sorbitol | 4.1 | standard-% |
| GPM | 47.7 | standard-% |
| GPS | 45.1 | standard-% |
| GPM + GPS | 92.9 | standard-% |

Refr. Means Refractometric.

Table 29 shows the results of the trials carried out. There is an increase in the free-flowability time of the crystalline isomalt type 1 (control) compared to the two water-containing crystalline isomalt starting preparations coated with syrup from trials 1 and 2. After application of the fine crystalline isomalt according to the invention in trial 3, the free-flowability time decreases again.

TABLE 29
Results of the control trials and the trial according to the invention

| | Free-flowability | Sorbitol | Moisture |
|---|---|---|---|
| Trial/No. | s/100 g | g/100 gTS | g/100 g |
| crystalline Isomalt | 7.4 * | 0.09 | 3.3 |
| Type 1 | | | |
| 1 | 8.3 * | 0.13 | 3.8 |
| 2 | 8.9 * | 0.18 | 4.0 |
| 3 | 8.1 * | 0.17 | 3.9 |

* with 10 mm nozzle

The free-flowability was determined according to EUROPEAN PHARMACOPOEIA 10.0 Method 2.9.16. Flowability.

Table 29 shows that there is a correlation in the addition of the isomalt solution to the preparation of the water-containing crystalline isomalt starting preparation with regard to free-flowability and water content. A deterioration in free-flowability compared to the crystalline control is observed. The free-flow time is extended by up to 20% due to the syrup film coating, depending on the proportion of syrup. The water content also increases by approx. 17% due to the syrup film coating with isomalt syrup.

In order to prove successful syrup film coating of the crystalline isomalt with the syrup, the control and the trial samples were tested for sorbitol content, which occurs to a greater extent in the isomalt syrup used. As described in the product analysis of the starting materials, the isomalt syrup has a sorbitol content of 4.1 standard-%, while crystalline isomalt type 1 has only 0.09 g/DM (standard-% and g/100 gDM comparable to first approximation).

Table 29 shows that the sorbitol content also increases as the proportion of syrup increases. The sorbitol content of the sample coated with syrup (9.6%) was twice as high as that of the isomalt-type control. Thus, a successful syrup film coating could be confirmed.

It could be shown that the addition of fine-grained isomalt particles surprisingly does not worsen the free-flowability of syrup film-coated isomalt crystals by, for example, baking, but rather significantly improves it.

1. A process for preparing a dried carbohydrate preparation from a water-containing crystalline carbohydrate starting preparation, comprising the process steps:
a) providing a water-containing crystalline carbohydrate starting preparation having 0.4 to 22.5 wt.-%, in particular 2.5 to 12 wt.-% water (each based on the total weight of the carbohydrate starting preparation), wherein the carbohydrate starting preparation has a content of water in excess of any possible water of crystallisation present and wherein the carbohydrate starting preparation is selected from the group consisting of a sucrose-containing composition, an isomaltulose- and trehalulose-containing composition and an isomalt-containing composition in a drying reactor, and carbohydrate particles, wherein the carbohydrate particles have a proportion of carbohydrate crystals with a diameter of less than or equal to 100 μm of at least 80 wt.-%,
b) metering in the carbohydrate particles via a metering device assigned to the drying reactor into the water-containing crystalline carbohydrate starting preparation presented in the drying reactor in an amount of 2 to 30 wt.-% (based on the total weight of the carbohydrate starting preparation) at a pressure of 10 to 1100 mbar, wherein the water-containing carbohydrate starting preparation presented in the drying reactor has a temperature of 20 to 80° C., in particular 40 to 60° C., and
c) homogenising the obtained mixture by means of a mixing device under the conditions mentioned in process step b) to obtain a dried carbohydrate preparation.

2. The process of claim 1, wherein the water-containing crystalline carbohydrate starting preparation having 0.4 to 22.5 wt.-%, in particular 2.5 to 12 wt.-% water (each based on the total weight of the carbohydrate starting preparation) provided in process step a) has liquid-film-coated crystalline carbohydrate particles and in process step c) a dried carbohydrate preparation is obtained from liquid-film-freed crystalline carbohydrate particles.

3. The process of claim 1 wherein the process is carried out continuously, semi-continuously or batchwise.

4. The process of claim 1, wherein the water-containing carbohydrate starting preparation comprises at least one poorly-soluble carbohydrate with a solubility at 20° C. in water of 5 to 53 g/100 g, in particular 1,1-GPM, isomaltulose, glucose or mannitol, and at least one well-soluble carbohydrate with a solubility at 20° C. in water of more than 53 g/100 g, in particular sucrose, 1,6-GPS, fructose, trehalulose or sorbitol.

5. The process of claim 1, wherein the water-containing carbohydrate starting preparation is a water-containing sucrose-containing composition having crystalline sucrose and at least one further carbohydrate-containing component selected from the group consisting of invert sugar, caramel sugar syrup and cane sugar syrup.

6. The process of claim 1, wherein the carbohydrates of the water-containing carbohydrate starting preparation and the carbohydrate particles are the same.

7. The process of claim 1, wherein the carbohydrate particles are metered in in process step b) in an amount of 2 to 25 wt.-%, in particular 4 to 20 wt.-%, in particular 4 to 15 wt.-% and in particular 4 to 10 wt.-% (each based on the total weight of the carbohydrate starting preparation).

8. The process of claim 1, wherein the metering-in of the carbohydrate particles in process step b) is carried out in a drying reactor free from excess pressure and negative pressure under atmospheric pressure, in a vacuum drying reactor under reduced pressure, in particular 10 to 900 mbar, in particular 20 to 600, in particular 30 to 400 mbar, in particular 40 to 200 mbar, in particular 50 to 100 mbar or in particular 600 to 800 mbar, in particular 650 to 750 mbar, in particular 700 mbar, or is carried out in a pressure drying reactor under a pressure of atmospheric pressure up to 1100 mbar.

9. The process of claim 1, wherein, following process step c), conditioning is carried out in a process step d).

10. The process of claim 1, wherein the conditioning according to process step d) is carried out at a temperature of at least 30° C., in particular 30 to 180° C., in particular 35 to 160° C., in particular 45 to 100° C., in particular 35 to 60° C., in particular 40 to 60° C., in particular 50 to 60° C.

11. The process of claim 1, wherein the conditioning according to process step d) is carried out at a pressure of 10 to 1100 mbar, in particular 10 to 900 mbar, in particular 20 to 600 mbar, in particular 30 to 400 mbar, in particular 40 to 200 mbar, in particular 50 to 100 mbar.

12. The process of claim 1, wherein the water-containing sucrose-containing starting preparation was recovered from sugar cane.

13. The process of claim 1, wherein the water-containing isomalt-containing starting preparation has a 1,1-GPM (1-O-alpha-D-glucopyranosyl-D-mannitol) and 1,6-GPS (6-O-alpha-D-glucopyranosyl-D-sorbitol) content of 95.0 to 100.0 wt.-% (based on the total dry mass of the water-containing isomalt-containing starting preparation).

14. The process of claim 1, wherein the water-containing isomalt-containing starting preparation has a 1,1-GPM (1-O-alpha-D-glucopyranosyl-D-mannitol) content of 45.0 to 50.0 wt.-% and a 1,6-GPS (6-O-alpha-D-glucopyranosyl-D-sorbitol) content of 50 to 55 wt.-% (each based on the total dry mass of the water-containing isomalt-containing starting preparation).

15. The process of claim 1, wherein the water-containing isomalt-containing starting preparation has a 1,1-GPM (1-O-alpha-D-glucopyranosyl-D-mannitol) content of 20.0 to 30.0 wt.-% and a 1,6-GPS (6-O-alpha-D-glucopyranosyl-D-sorbitol) content of 70.0 to 80.0 wt.-% (each based on the total dry mass of the water-containing isomalt-containing starting preparation).

16. The process of claim 1, wherein the water-containing isomalt-containing starting preparation has 1,1-GPS (1-O-alpha-D-glucopyranosyl-D-sorbitol), sorbitol, mannitol, or GPI (glucopyranosyl-iditol) or a mixture of two or more thereof.

17. The process of claim 1, wherein the water-containing isomaltulose- and trehalulose-containing starting preparation has fructose, glucose, isomaltose or isomelecitose or a mixture of two or more thereof.
